(19) United States
(12) Patent Application Publication
(10) Pub. No.: US 2024/0424306 A1
(43) Pub. Date: Dec. 26, 2024

(54) SENSOR ANCHOR LOOP CONFIGURATIONS FOR LODGING AN IMPLANTABLE WIRELESS SENSOR IN A LUMEN

(71) Applicant: TC1 LLC, St. Paul, MN (US)

(72) Inventors: Lindsay Hall, Atlanta, GA (US); Dhruv Ranjan, Naperville, IL (US); Tracee Eidenschink, Wayzata, MN (US); Suraj Vuduta, Alpharetta, GA (US); Lauren Clark, Brookhaven, GA (US)

(21) Appl. No.: 18/664,443

(22) Filed: May 15, 2024

Related U.S. Application Data
(60) Provisional application No. 63/509,111, filed on Jun. 20, 2023.
(60) Provisional application No. 63/509,116, filed on Jun. 20, 2023.

(51) Int. Cl. A61N 1/365; A61N 1/05; A61N 1/362; A61N 1/368; A61N 1/375

(52) U.S. Cl. CPC A61N 1/36564; A61N 1/0563; A61N 1/3622; A61N 1/36842; A61N 1/37518

(57) ABSTRACT

An implantable wireless sensor that is configured for deploying within a lumen in a body comprises a sensor body and a first anchoring element. The sensor body comprises a proximal end and a distal end. The first anchoring element is coupled to the proximal end of the sensor body and a second anchoring element is coupled to the distal end of the sensor body. At least one of the first and second anchoring elements are configured to lodge the sensor body within the lumen. The first and second anchoring elements are asymmetric by i) size with respect to each other prior to releasably retaining the sensor in a fixed relationship with a delivery system or ii) number of elements.

### CROSS-REFERENCE TO RELATED APPLICATIONS

This application claims priority to U.S. Provisional Patent Application No. 63/509,111, filed 20-June-2023, entitled “SENSOR ANCHOR LOOP CONFIGURATIONS FOR LODGING AN IMPLANTABLE WIRELESS SENSOR IN A LUMEN” and U.S. Provisional Patent Application No. 63/509,116, filed 20-June-2023, entitled “SENSOR ANCHOR LOOP CONFIGURATIONS FOR LODGING AN IMPLANTABLE WIRELESS SENSOR IN A LUMEN”, the subject matter of which are incorporated herein by reference in their entireties.

### BACKGROUND

Embodiments of the present disclosure generally relate to methods and devices for anchoring an implantable wireless sensor at a desired location within a body, as well as attaching the sensor to, and releasing the sensor from, a device delivery system.

Implantable medical sensors are currently available to monitor certain physiologic conditions, such as blood pressure. The size of the implantable medical sensor is limited due to target implant locations within the patient, such as within blood vessels or lumens. One example of an implantable medical sensor is a pulmonary arterial pressure (PAP) sensor. In some cases, the sensors can be passive, utilizing an external device located outside of the patient body for supplying energy to power the generation and/or communication of the physiological data. In other cases, the sensors may have an onboard battery capable of limited functionality.

One example of an implantable sensor is one that has a distal anchor loop and a proximal anchor loop. The distal and proximal anchor loops are the same size and provide the ability to secure the sensor to the delivery system. The distal and proximal anchor loops can utilize nitinol self-expanding loops that expand upon release from the delivery system to secure and/or anchor the sensor in the anatomy.

Although the current sensor design can simplify the assembly and useability, there are many different anatomical areas where sensors may eventually be placed. For example, some anatomical areas may include structure having different size(s) and/or shapes(s) that receive the distal and proximal anchor loops, resulting in only one of the loops fully engaging the anatomy. This lack of engagement with the lumen can result in migration of the sensor.

A need remains for methods and devices that improve the delivery and release of the implantable device, as well as the retention of the implantable device within the anatomy to reduce migration of the sensor.

### SUMMARY

In accordance with embodiments herein, an implantable wireless sensor for deploying within a lumen in a body comprises a sensor body comprising a proximal end and a distal end, and a first anchoring element coupled to the proximal end of the sensor body and a second anchoring element coupled to the distal end of the sensor body. At least one of the first and second anchoring elements are configured to lodge the sensor body within the lumen, wherein the first and second anchoring elements are asymmetric by i) size with respect to each other prior to releasably retaining the sensor in a fixed relationship with a delivery system or ii) number of elements.

Optionally, the first anchoring element coupled to the proximal end of the sensor body is relatively larger than the second anchoring element coupled to the distal end of the sensor body. Optionally, the first anchoring element coupled to the proximal end of the sensor body spans a larger diameter than the second anchoring element, wherein the first anchoring element is configured to lodge in a relatively larger lumen and the second anchoring element is configured to lodge in a relatively smaller lumen.

Optionally, the second anchoring element comprises two elements, wherein the two elements comprise first and second loops, wherein the first loop is oriented to extend at a non-zero angle with respect to at least one of horizontal and vertical planes associated with the sensor body, wherein each of the first and second loops span smaller diameters than the first anchoring element to lodge in second and third lumens extending from the first lumen that are relatively smaller than the first lumen. Optionally, the first anchoring element is a body anchoring loop and the second anchoring element is a distal anchoring loop, wherein the body anchoring loop is relatively longer than the distal anchoring loop, the body anchoring loop and distal anchoring loop extending distally with respect to the sensor body, a distal end of the body anchoring loop extending under the distal anchoring loop, wherein the body anchoring loop is configured to engage the lumen proximate opposite first and second side surfaces of the sensor.

Optionally, the first anchoring element is a body anchoring loop and the second anchoring element is a proximal anchoring loop and wherein the body anchoring loop is relatively longer than the proximal anchoring loop. The body anchoring loop and proximal anchoring loop extend proximally with respect to the sensor body. A proximal end of the body anchoring loop extends under the proximal anchoring loop, and wherein the body anchoring loop is configured to engage the lumen proximate opposite first and second side surfaces of the sensor. Optionally, the first and second anchoring elements are configured to extend coplanar with proximal and distal directions. Optionally, the first and second anchoring elements include distal portions proximate the distal end of the sensor body, and the sensor further comprises a restraining feature coupled to the distal end of the sensor body, the restraining feature configured to restrain the distal portions of the first and second anchoring elements.

In accordance with embodiments herein, an implantable wireless sensor for deploying within a lumen in a body comprises a sensor body comprising a proximal end and a distal end, and a first anchoring element coupled to the proximal end of the sensor body and a second anchoring element coupled to the distal end of the sensor body. At least one of the first and second anchoring elements is configured to lodge the sensor body within the lumen. The first anchoring element comprises an anchoring body extending in a proximal direction with respect to the proximal end of the sensor body. The anchoring body includes at least one integrated attachment feature configured to retain a release element associated with a delivery system. The release element is configured to releasably retain the sensor in a fixed relationship with a delivery system in advance of deployment of the sensor in a body.

Optionally, the at least one integrated attachment feature is a loop. Optionally, the at least one integrated attachment feature comprises first, second, and third integrated attachment features configured to retain the release element. The first and second integrated attachment features are positioned on opposite sides of the anchoring body, and the first and second integrated attachment features are positioned to overlap each other when retained by the release element.

In accordance with embodiments herein, an implantable wireless sensor for deploying within a lumen in a body comprises a sensor body comprising a distal end and a proximal end and an anchoring element. The sensor body further comprises opposite first and second side surfaces extending between the distal end and the proximal end. The anchoring element is coupled to a proximal end of the sensor body and configured to extend in a distal direction along the opposite first and second side surfaces. The anchoring element extends beyond the distal end of the sensor, wherein the anchoring element is configured to engage the lumen proximate the first and second side surfaces.

Optionally, the anchoring element is a loop that curves to form first and second inward bends along each of the first and second side surfaces. The first and second inward bends are configured to extend away from walls of the lumen, and wherein the first and second inward bends are formed distal of the distal end of the sensor body. A distance between the first and second inward bends is less than a distance between portions of the anchoring element extending along the first and second side surfaces. Optionally, side portions of the loop that extend distally of the first and second inward bends are configured to engage the lumen. Optionally, the anchoring element is a loop, the loop having a cross-over formed distally with respect to the distal end to form a second loop, the second loop configured to engage the lumen along the first and second side surfaces. Optionally, the anchoring element is a loop, and the sensor further comprises a restraining feature coupled to the distal end of the sensor body. The restraining feature restrains the anchoring element and forms a distal loop and a body loop, wherein the body loop is configured to expand when squeezing pressure is applied to the distal loop.

In accordance with embodiments herein an implantable wireless sensor for deploying within a lumen in a body comprises a sensor body comprising a distal end and a proximal end, and first and second anchoring elements. The sensor body further comprises opposite first and second side surfaces extending between the distal end and the proximal end and opposite top and bottom surfaces extending between the distal end and the proximal end. The first anchoring element is coupled to the proximal end of the sensor body. The second anchoring element is coupled to the distal end of the sensor body, wherein the first and second anchoring elements are configured to be releasably attached to a delivery system with a release element and held in a planar configuration along the delivery system. Responsive to the removal of the release element, the first anchoring element is configured to pivot away from the sensor body proximate the top surface and the second anchoring element is configured to pivot away from the sensor body proximate to the bottom surface. The first and second anchoring elements are configured to engage opposite sides of the lumen proximate the distal and proximal ends to keep the sensor body centered within the lumen.

Optionally, the first and second anchoring elements are loops, fingers, or shaped to provide two points of contact with the lumen. Optionally, a third anchoring element is coupled to the proximal end of the sensor body, and a fourth anchoring element is coupled to the distal end of the sensor body. Responsive to the removal of the release element, the third anchoring element is configured to pivot away from the sensor body proximate the top surface and the second anchoring element is configured to pivot away from the sensor body proximate to the bottom surface. The third and fourth anchoring elements are configured to engage opposite sides of the lumen proximate the proximal and distal ends to keep the sensor body centered within the lumen.

In accordance with embodiments herein, an implantable wireless sensor for deploying within a lumen in a body comprises a sensor body comprising a distal end and a proximal end, and first, second, and third anchoring elements. The sensor body further comprises opposite first and second side surfaces extending between the distal end and the proximal end and opposite top and bottom surfaces extending between the distal end and the proximal end. The first anchoring element is coupled to one of the surfaces of the sensor body and the second anchoring element coupled to one of the surfaces of the sensor body. The third anchoring element is coupled to one of the surfaces proximate the distal end of the sensor body, wherein the first, second, and third anchoring elements are configured to be releasably retained by a delivery system with a release element and held, by the release element, in a planar configuration along the delivery system. Responsive to the removal of the release element, the first, second, and third anchoring elements are configured to pivot away from the sensor body. The first and second anchoring elements are configured to engage opposite sides of the lumen and keep the sensor body centered within the lumen. The third anchoring element is configured to pivot away from the sensor body and engage the lumen distal of the sensor body.

Optionally, the first and second anchoring elements are coupled to the sensor body within a central region of the sensor body. Optionally, a fourth anchoring element is coupled to one of the surfaces proximate the proximal end of the sensor body, wherein the fourth anchoring element is configured to be releasably retained by the delivery system with the release element and held, by the release element, in the planar configuration along the delivery system. Responsive to the removal of the release element, the fourth anchoring element is configured to pivot away from the sensor body and engage the lumen proximal of the sensor body, wherein the release element is a tether.

Optionally, the sensor further comprising a fourth anchoring element coupled to one of the surfaces proximate the proximal end of the sensor body, wherein the fourth anchoring element is configured to be releasably attached to the release element and is configured to be delivered through a sheath. Responsive to the removal of the sheath, the fourth anchoring element is configured to engage the lumen proximal of the sensor body. Optionally, the fourth anchoring element is configured to be withdrawn into the sheath after the release element has released the fourth anchoring element. Optionally, responsive to a second removal of the sheath, the fourth anchoring element is configured to engage the lumen proximal of the sensor body. Optionally, wherein the first, second, and third anchoring elements are loops, fingers, or shaped to provide two points of contact with the lumen.

In accordance with embodiments herein, an implantable wireless sensor for deploying within a lumen in a body comprises a sensor body comprising a proximal end, a distal end, and a first anchoring element. The first anchoring element is coupled to the distal end of the sensor body and is configured to lodge the sensor body within the lumen. The first anchoring element comprises a loop having a first diameter when the first anchoring element is freely released and not subject to any bias. Responsive to a force moving the sensor body in a distal direction within the lumen after the sensor is deployed within the lumen, the first anchoring element is configured to alter shape to a second diameter that is less than the first diameter. Responsive to the sensor body moving in a proximal direction within the lumen after the sensor is deployed within the lumen, the first anchoring element is configured to alter shape to a third diameter that is greater than the first diameter.

Optionally, the first anchoring element is shaped to form two curved anchor points facing in the proximal direction when having the first diameter. Optionally, the two curved anchor points are positioned to contact opposite sides of the lumen. Optionally, the first anchoring element is shaped to form at least four curved anchor points facing in the proximal direction when having the first diameter, wherein a first portion of the at least four curved anchor points are positioned to contact a first side of the lumen and a second portion of the at least four curved anchor points are positioned to contact a second side of the lumen that is opposite the first side of the lumen.

Optionally, the sensor further comprises a second anchoring element is coupled to the proximal end of the sensor body. The second anchoring element is configured to lodge the sensor body within the lumen. The second anchoring element comprises a loop having a fourth diameter when the second anchoring element is lodged within the lumen, wherein the second anchoring element is shaped to form at least two curved anchor points facing in the distal direction. Optionally, responsive to the sensor body moving in the proximal direction within the lumen after the sensor is deployed within the lumen, the second anchoring element is configured to alter shape to a fifth diameter that is less than the fourth diameter, and responsive to the sensor body moving in the distal direction within the lumen after the sensor is deployed within the lumen, the second anchoring element is configured to alter shape to a sixth diameter that is greater than the fourth diameter.

In accordance with embodiments herein, an implantable wireless sensor for deploying within a lumen in a body comprises a sensor body comprising a proximal end and a distal end and an anchoring element. The anchoring element is attached to the proximal end of the sensor body. The anchoring element comprises a rod having a distal end attached to the proximal end of the sensor body and a proximal end configured to be removably attached to a delivery rod of a delivery system. The rod has a length extending between the proximal and distal ends of the rod. At least two loops are attached to a proximal end of the rod, the at least two loops configured to lodge the sensor body within the lumen when deployed by the delivery system.

Optionally, the proximal end of the rod is removably attached to the delivery rod with a threaded fastener. Optionally, the at least two loops are configured to compress and tighten around the rod when contained within a sheath, and wherein the at least two loops are configured to expand outwardly away from the rod and lodge the sensor within the lumen when the sheath is retracted in a proximal direction beyond the proximal end of the rod. Optionally, the at least two loops include a first group having at least one loop configured to extend in a proximal direction and a second group having at least one loop configured to extend in a distal direction when the sheath is retracted in the proximal direction beyond the proximal end of the rod.

In accordance with embodiments herein, an implantable wireless sensor for deploying within a lumen in a body comprises a sensor body comprising a distal end and a proximal end, a first anchoring element, and a radiopaque marker. The first anchoring element is coupled to one of the distal end or the proximal end of the sensor body. The first anchoring element has an anchoring body extending away from the sensor body, and the first anchoring element is configured to lodge the sensor body within the lumen. The radiopaque marker is integrated with the anchoring body.

Optionally, the radiopaque marker is attached at a farthest position of the first anchoring element away from the sensor body. Optionally, the radiopaque marker includes an integrated attachment feature configured to releasably retain a release element associated with a delivery system, wherein the release element is configured to releasably retain the integrated attachment feature to retain the sensor in a fixed relationship with the delivery system in advance of deployment of the sensor in the lumen.

Optionally, the radiopaque marker includes a hole, the anchoring body extending through the hole. Optionally, the radiopaque marker includes a hole configured to receive a release element of a delivery system. Optionally, the radiopaque marker is attached to the anchoring body by swaging or crimping. Optionally, the radiopaque marker comprises a radiopaque material extending over at least a portion of the anchoring body, wherein the anchoring body includes shape memory material. Optionally, the radiopaque marker comprises a radiopaque core extending through the anchoring body, the radiopaque core at least partially surrounded by a shape memory material. Optionally, the radiopaque marker is a loop.

In accordance with embodiments herein, an implantable wireless sensor for deploying within a lumen in a body, comprises a sensor body comprising a distal end and a proximal end, the sensor body further comprising opposite first and second side surfaces extending between the distal end and the proximal end, opposite top and bottom surfaces extending between the distal end and the proximal end, and opposite first and second end surfaces, wherein the first side surface is adjacent along a first side with the top surface and adjacent along a second side with the bottom surface, wherein the second side surface is adjacent along a first side with the top surface and adjacent along a second side with the bottom surface. The sensor further comprises a first anchoring element coupled to the first side surface within a central region of the sensor body and a second anchoring element coupled to one of the top or bottom surfaces within a central region of the sensor body. Responsive to deploying the sensor within the lumen with a delivery system, the first anchoring element is configured to pivot away from the sensor body to engage a first side of the lumen and the second anchoring element is configured to pivot away from the sensor body to engage a second side of the lumen that is adjacent to the first side of the lumen, the first and second anchoring elements configured to keep the sensor body off-center within the lumen.

Optionally, the sensor further comprises third and fourth anchoring elements coupled to the first and second end surfaces, respectively, wherein the third and fourth anchoring elements are configured to be releasably attached to the delivery system with a release element. Optionally, the sensor further comprises a fifth anchoring element coupled to the second side surface within the central region of the sensor body, and a sixth anchoring element coupled to one of the top or bottom surfaces within the central region of the sensor body that is opposite with respect to the second anchoring element. Responsive to deploying the sensor with the lumen with the delivery system, the fifth anchoring element is configured to pivot away from the sensor body to engage a third side of the lumen and the sixth anchoring element is configured to pivot away from the sensor body to engage a fourth side of the lumen that is adjacent to the third side of the lumen, the first, second, third, and fourth anchoring elements configured to keep the sensor body centered within the lumen.

### BRIEF DESCRIPTION OF THE DRAWINGS

FIG. 1A illustrates an example of pulmonary branches having a sensor with asymmetric anchoring elements lodged therein in accordance with embodiments herein.

FIG. 1B illustrates a top view showing the sensor and anchoring elements of FIG. 1A in accordance with embodiments herein.

FIGS. 1C and 1D illustrate top views of the sensor having an anchoring element coupled to the proximal end of the sensor body and an anchoring element that includes a plurality of elements coupled to the distal end in accordance with embodiments herein.

FIG. 1E shows the sensor of FIG. 1C having the anchoring elements lodged within a lumen proximate to where the lumen bifurcates in accordance with embodiments herein.

FIG. 2A is an isometric view of a sensor having anchoring elements coupled to the proximal and distal ends of the sensor body that both project in the distal direction in accordance with embodiments herein.

FIG. 2B shows a top view of the configuration of FIG. 2A.

FIGS. 2C and 2D show top views of a sensor having a single anchoring element in accordance with embodiments herein.

FIG. 2E illustrates a restraining feature that retains the anchoring element(s) proximate one end of the sensor body in accordance with embodiments herein.

FIG. 3 illustrates a sensor having an anchoring element including a plurality of open-sided curved fingers attached to a spline in accordance with embodiments herein.

FIG. 4A shows a cross-sectional view of a sensor that has an anchoring element that folds in on itself when interconnected with a sheathed delivery system in accordance with embodiments herein.

FIG. 4B shows a cross-sectional view of a push rod and interlocking feature of FIG. 4A in accordance with embodiments herein.

FIG. 4C shows a cross-sectional view wherein a distal portion of an inner sheath of the delivery system has been advanced out of the outer sheath in accordance with embodiments herein.

FIG. 4D shows a cross-sectional view wherein the anchoring element of the sensor has been fully advanced out of the inner sheath in accordance with embodiments herein.

FIG. 5A is a side view of a sensor body that has anchoring elements configured to engage opposite sides of the lumen to position the sensor body within the lumen in accordance with embodiments herein.

FIG. 5B is a top view wherein the anchoring elements have pivoted away from the sensor body to engage opposite sides of the lumen in accordance with embodiments herein.

FIG. 5C shows an example wherein anchoring elements are releasably attached to a delivery shaft (not shown) using one or more tethers in accordance with embodiments herein.

FIGS. 5D and 5E show side and isometric views of the sensor of FIG. 5C deployed within the lumen in accordance with embodiments herein.

FIGS. 5F and 5G show side and top views of the sensor having an asymmetric number of anchoring elements coupled to opposite ends of the sensor body in accordance with embodiments herein.

FIGS. 5H and 51 are top views of the sensor having a portion of the anchoring elements coupled to a central region of the sensor body in accordance with embodiments herein.

FIG. 5J shows a top view of a sensor having anchoring elements with rounded points configured to engage the wall of the lumen in accordance with embodiments herein.

FIGS. 6A and 6B illustrate isometric views of a sensor having an anchoring element that includes at least one integrated attachment feature in accordance with embodiments herein.

FIGS. 6C and 6D show the sensor having the attachment features formed integral with the anchoring element, wherein the attachment features extend inwardly in accordance with embodiments herein.

FIGS. 6E and 6F show the sensor of FIGS. 6C and 6D, respectively, wherein at least two of the attachment features overlap each other when force is applied to compress the anchoring element in accordance with embodiments herein.

FIG. 6G is a view of the anchoring element of FIG. 6B retained by a tether to a delivery system in accordance with embodiments herein.

FIG. 6H shows a coiled ribbon shaped anchoring element that can be retained by a tether in accordance with embodiments herein.

FIG. 6I shows the sensor having the coiled ribbon anchoring element of FIG. 6H prior to or in advance of being deployed within a lumen in accordance with embodiments herein.

FIG. 6J shows that the end of the coiled ribbon anchoring element can be cut or shaped to improve the fit into the skive in accordance with embodiments herein.

FIG. 7A is an isometric view of the sensor having anchoring elements attached to multiple surfaces in accordance with embodiments herein.

FIG. 7B is a top view of a sensor having anchoring elements coupled to opposite side surfaces in accordance with embodiments herein.

FIG. 7C is an isometric view of a sensor having anchoring elements coupled to opposite sides in accordance with embodiments herein.

FIG. 8A is a top view of a sensor having anchoring elements coupled to proximal and distal ends of the sensor body that are configured to alter shape, diameter and/or length to resist or facilitate movement within a lumen in accordance with embodiments herein.

FIG. 8B is a top view of an anchoring element that has altered shape to have an increased diameter upon movement within a lumen in accordance with embodiments herein.

FIG. 8C is a top view of a sensor having anchoring elements that are configured to alter shape as a result of movement in accordance with embodiments herein.

FIGS. 8D, 8E, and 8F are top views of an anchoring element with different diameters that is configured to alter shape upon movement within a lumen in accordance with embodiments herein.

FIGS. 8G and 8H are top views of anchoring elements that are configured to alter shape and that have more than two anchor points configured to contact the walls of the lumen in accordance with embodiments herein.

FIG. 9A shows an anchoring element that includes a rod and at least two loops attached to a proximal end of the rod that can be used as a snare to deliver or retrieve the sensor in accordance with embodiments herein.

FIG. 9B shows the sensor of FIG. 9A deployed within the lumen.

FIG. 9C shows the sensor of FIG. 9A wherein the sensor includes a first group of loops oriented to generally extend in the proximal direction and a second group of loops oriented to generally extend in the distal direction in accordance with embodiments herein.

FIG. 10A shows an isometric view of a sensor having a radiopaque marker integrated with an anchoring element in accordance with embodiments herein.

FIG. 10B is an isometric view of a sensor having multiple radiopaque markers attached to the anchoring element in accordance with embodiments herein.

FIG. 10C is a side view of a sensor releasably attached to a tethered delivery system having attachment features and radiopaque markers in accordance with embodiments herein.

FIG. 10D is a side view of a sensor having radiopaque markers integrated with the anchoring elements in accordance with embodiments herein.

FIG. 10E is a top view of a sensor having different radiopaque markers integrated with anchoring elements in accordance with embodiments herein.

FIG. 10F shows a view wherein the hole orientation of the integrated attachment feature that releasably retains the tether is approximately in the same plane as the loop that interfaces with the anchoring element and another view wherein the hole orientation is twisted, such as to optimize the profile to the delivery system, in accordance with embodiments herein.

FIG. 10G shows a view of a sensor with a radiopaque coupler and an attachment feature integrated with the anchoring element in accordance with embodiments herein.

FIGS. 10H and 10I show coupled and uncoupled views, respectively, of a sensor that has a two-part anchoring element joined with a radiopaque coupler in accordance with embodiments herein.

FIG. 10J is a cross-sectional view of a wire that has a radiopaque core that is used to form an anchoring element in accordance with embodiments herein.

FIG. 11 illustrates a system that includes an implantable medical device (IMD), an implantable sensor, and an external device implemented in accordance with embodiments herein.

FIG. 12 is an exemplary tandem delivery system.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

Embodiments may be implemented in connection with concepts describe in the following patents, all of which are expressly incorporated in their entireties by reference: U.S. Pat. No. 10,653,859, titled “Delivery Catheter Systems and Methods” having an issue date of May 19, 2020, U.S. Pat. No. 10,894,144, titled “Apparatus and method for sensor deployment and fixation” having an issue date of Jan. 19, 2021, U.S. Pat. No. 11,179,048, titled “System for deploying an implant assembly in a vessel” having an issue date of Nov. 23, 2021; and U.S. patent application Ser. No. 17/820,654 entitled “System and Method for Intra-Body Communication of Sensed Physiologic Data”, filed on Aug. 18, 2022, which is incorporated herein by reference in its entirety.

Embodiments herein may be implemented in connection with the concepts described in the following patents and applications, all of which are expressly incorporated in their entireties by reference: U.S. Pat. No. 10,433,764B2, titled “Implantable sensor enclosure with thin sidewalls” having an issue date of Oct. 9, 2019; U.S. Pat. No. 9,867,552, titled “Implantable sensor enclosure with thin sidewalls” having an issue date of Sep. 16, 2018; U.S. Pat. No. 9,538,958B2, titled “Permittivity shielding” having an issue date of Jan. 10, 2017; U.S. Pat. No. 8,894,582B, titled “Cardiac pressure monitoring device” having an issue date of Nov. 25, 2014;U.S. Pat. No. 7,677,107B2, titled “Wireless pressure sensor and method for fabricating wireless pressure sensor for integration with an implantable device” having an issue date of Mar. 16, 2010; U.S. patent application Ser. No. 20/200,260991A1, titled “Pressure sensing implant” having a publication date of Aug. 20, 2020; U.S. patent application Ser. No. 20/190,200928A1, titled “Pressure sensing implant” having an issue date of Jul. 4, 2019; U.S. patent application Ser. No. 20/180,116552A1, titled “IMPLANTABLE SENSOR ENCLOSURE WITH THIN SIDEWALLS” having a publication date of May 3, 2018; U.S. patent application Ser. No. 20/160,324443A1, titled “PRESSURE SENSING IMPLANT” having a publication date of Nov. 10, 2016; U.S. patent application Ser. No. 20/160,029956A1, titled “PRESSURE SENSING IMPLANT” having a publication date of Feb. 4, 2016; U.S. patent application Ser. No. 20/140,155710A1, titled “IMPLANTABLE SENSOR ENCLOSURE WITH THIN SIDEWALLS” having an issue date of Jun. 5, 2014; U.S. patent application Ser. No. 20/090,024042A1, titled “METHOD AND SYSTEM FOR MONITORING VENTRICULAR FUNCTION OF A HEART” having a publication date of Jan. 22, 2009; U.S. Pat. No. 20,090,007679A1, titled “WIRELESS PRESSURE SENSOR AND METHOD FOR FABRICATING WIRELESS PRESSURE SENSOR FOR INTEGRATION WITH AN IMPLANTABLE DEVICE” having an issue date of Jan. 8, 2009; U.S. patent application Ser. No. 20/090,011117A1 titled “METHODS FOR TEXTURING A SURFACE OF AN ENDOVASCULAR IMPLANT” having a publication date of Jan. 8, 2009; U.S. Pat. No. 8,382,677 titled “An anchored implantable pressure monitor” having an issue date of Feb. 26, 2013; U.S. Pat. No. 8,355,777 titled “Apparatus and method for sensor deployment and fixation” having an issue date of Jan. 15, 2013; U.S. Pat. No. 8,353,841 titled “Apparatus and method for sensor deployment and fixation” having an issue date of Jan. 15, 2013; U.S. Pat. No. 8,118,749 titled “Apparatus and method for sensor deployment and fixation” having an issue date of Feb. 21, 2012; U.S. Pat. No. 8,021,307 titled “Apparatus and method for sensor deployment and fixation” having an issue date of Sep. 20, 2011; U.S. Pat. No. 7,966,886 titled “Method and apparatus for measuring pressure inside a fluid system” having an issue date of Jun. 28, 2011; U.S. Pat. No. 7,936,174 titled “Coupling loop” having an issue date of May 3, 2011; U.S. Pat. No. 7,595,647 titled “Cable assembly for a coupling loop” having an issue date of Sep. 29, 2009; U.S. Pat. No. 7,432,723 titled “Coupling loop” having an issue date of Oct. 7, 2008; U.S. Pat. No. 8,111,150 titled “Physiological data acquisition and management system for use with an implanted wireless sensor” having an issue date of Feb. 7, 2012; U.S. Pat. No. 8,159,348 titled “Communication system with antenna box amplifier” having an issue date of Apr. 17, 2012; U.S. Pat. No. 8,237,451 titled “Communicating with an implanted wireless sensor” having an issue date of Aug. 7, 2012; U.S. Pat. No. 7,839,153 titled “Communicating with an implanted wireless sensor” having an issue date of Nov. 23, 2010; U.S. Pat. No. 7,679,355 titled “Communicating with an implanted wireless sensor” having an issue date of Mar. 16, 2010; U.S. Pat. No. 7,550,978 titled “Communicating with an implanted wireless sensor” having an issue date of Jun. 23, 2009; U.S. Pat. No. 7,498,799B2 titled “Communicating with an implanted wireless sensor” having an issue date of Mar. 3, 2009; U.S. Pat. No. 7,466,120B2 titled “Communicating with an implanted wireless sensor” having an issue date of Dec. 16, 2008; U.S. Pat. No. 7,439,723B2 titled “Communicating with an implanted wireless sensor” having an issue date of Oct. 21, 2008; U.S. Pat. No. 7,245,117B1 titled “Communicating with implanted wireless sensor” having an issue date of Jul. 17, 2007; U.S. Pat. No. 8,669,770B2 titled “Selectively actuating wireless, passive implantable sensor” having an issue date of Mar. 11, 2014; U.S. Pat. No. 8,159,348B2 titled “Communication system with antenna box amplifier” having an issue date of Apr. 17, 2012; U.S. Pat. No. 7,932,732B2 titled “Preventing a false lock in a phase lock loop” having an issue date of Apr. 26, 2011; U.S. Pat. No. 7,710,103B2 titled “Preventing false locks in a system that communicates with an implanted wireless sensor” having an issue date of May 4, 2010; U.S. Pat. No. 7,492,144 titled “Preventing false locks in a system that communicates with an implanted wireless sensor” having an issue date of Feb. 17, 2009; U.S. Pat. No. 10,143,388 titled “Method of manufacturing implantable wireless sensor for pressure measurement” having an issue date of Dec. 4, 2018;U.S. Pat. No. 9,792,469 titled “Wireless physical property sensor with digital communications” having an issue date of Oct. 17, 2017; U.S. Pat. No. 9,653,926 titled “Physical property sensor with active electronic circuit and wireless power and data transmission” having an issue date of May 16, 2017; U.S. Pat. No. 9,041,416 titled “Physical property sensor with active electronic circuit and wireless power and data transmission” having an issue date of May 26, 2015; U.S. Pat. No. 8,264,240 titled “Physical property sensor with active electronic circuit and wireless power and data transmission” having an issue date of Sep. 11, 2012; U.S. Pat. No. 7,621,036 titled “Method of manufacturing implantable wireless sensor for in vivo pressure measurement” having an issue date of Nov. 24, 2009; U.S. Pat. No. 7,574,792 titled “Method of manufacturing an implantable wireless sensor” having an issue date of Aug. 18, 2009; U.S. Pat. No. 8,360,984 titled “Hypertension system and method” having an issue date of Jan. 29, 2013; U.S. Pat. No. 8,264,240B2 titled “Physical property sensor with active electronic circuit and wireless power and data transmission” having an issue date of Sep. 11, 2012; U.S. Pat. No. 8,025,625 titled “Sensor with electromagnetically coupled hermetic pressure reference” having an issue date of Sep. 27, 2011; U.S. Pat. No. 7,909,770 titled “Method for using a wireless pressure sensor to monitor pressure inside the human heart” having an issue date of Mar. 22, 2011; U.S. Pat. No. 7,908,018 titled “Flexible electrode” having an issue date of Mar. 15, 2011; U.S. Pat. No. 7,854,172 titled “Hermetic chamber with electrical feedthroughs” having an issue date of Dec. 21, 2010; U.S. Pat. No. 7,662,653 titled “Method of manufacturing a hermetic chamber with electrical feedthroughs” having an issue date of Feb. 16, 2010; U.S. Pat. No. 7,647,836 titled “Hermetic chamber with electrical feedthroughs” having an issue date of Jan. 19, 2010; U.S. Pat. No. 7,829,363 titled “Method and apparatus for microjoining dissimilar materials” having an issue date of Nov. 9, 2010; U.S. Pat. No. 7,812,416 titled “Methods and apparatus having an integrated circuit attached to fused silica” having an issue date of Oct. 12, 2010; U.S. Pat. No. 7,748,277 titled “Hermetic chamber with electrical feedthroughs” having an issue date of Jul. 6, 2010; U.S. Pat. No. 7,699,059 titled “Implantable wireless sensor” having an issue date of Apr. 20, 2010; U.S. Pat. No. 7,667,547 titled “Loosely-coupled oscillator” having an issue date of Feb. 23, 2010; U.S. Pat. No. 7,481,771 titled “Implantable wireless sensor for pressure measurement within the heart” having an issue date of Jan. 27, 2009; U.S. Pat. No. 6,855,115 titled “Implantable wireless sensor for pressure measurement within the heart” having an issue date of Feb. 15, 2005; U.S. Pat. No. 7,147,604 titled “High Q factor sensor” having an issue date of Dec. 12, 2006; U.S. patent application Ser. No. 20/200,146562 titled “Sensor delivery system and method” having a publication date of May 14, 2020; U.S. patent application Ser. No. 20/150,208929 titled “PRESSURE SENSOR, ANCHOR, DELIVERY SYSTEM AND METHOD” having a publication date of Jul. 30, 2015; U.S. Pat. No. 8,382,677 titled “An anchored implantable pressure monitor” having an issue date of Feb. 26, 2013; U.S. Pat. No. 10,282,571 titled “Self test device and method for wireless sensor reader” having an issue date of May 7, 2019; U.S. Pat. No. 9,996,712 titled “Self test device and method for wireless sensor reader” having an issue date of Jun. 12, 2018; US Patent 20170061168 titled “SELF TEST DEVICE AND METHOD FOR WIRELESS SENSOR READER” having an issue date of Mar. 2, 2017; U.S. Pat. No. 10,003,862 titled “Wireless sensor reader” having an issue date of Jun. 29, 2018; U.S. Pat. No. 9,894,425 titled “Wireless sensor reader” having an issue date of Feb. 23, 2017; U.S. Pat. No. 9,721,463 titled “Wireless sensor reader” having an issue date of Aug. 1, 2017; U.S. Pat. No. 9,489,831 titled “Wireless sensor reader” having an issue date of Nov. 8, 2016; U.S. Pat. No. 9,305,456 titled “Wireless sensor reader” having an issue date of Apr. 5, 2016; U.S. Pat. No. 8,570,186 titled “Wireless sensor reader” having an issue date of Oct. 29, 2013; U.S. Pat. No. 8,493,187 titled “Wireless sensor reader” having an issue date of Jul. 23, 2013; U.S. Pat. No. 8,432,265 titled “Wireless sensor reader” having an issue date of Apr. 30, 2013; U.S. Pat. No. 8,154,389 titled “Wireless sensor reader” having an issue date of Apr. 10, 2012; U.S. Pat. No. 7,839,153 titled “Communicating with an implanted wireless sensor” having an issue date of Nov. 23, 2010; U.S. Pat. No. 7,679,355 titled “Communicating with an implanted wireless sensor” having an issue date of Mar. 16, 2010; U.S. patent application Ser. No. 20/170,309164 titled “WIRELESS SENSOR READER” having a publication date of Oct. 26, 2017; U.S. patent application Ser. No. 20/170,055048 titled “WIRELESS SENSOR READER” having a publication date of Feb. 23, 2017; U.S. patent application Ser. No. 20/160,210846 titled “WIRELESS SESOR READER” having a publication date of Jul. 21, 2016; U.S. patent application Ser. No. 20/140,306807 titled WIRELESS SENSOR READER having a publication date of Oct. 16, 2014; U.S. patent application Ser. No. 20/140,028467 titled “WIRELESS SENSOR READER” having a publication date of Jan. 30, 2014; U.S. patent application Ser. No. 20/130,222153 titled “WIRELESS SENSOR READER” having a publication date of Aug. 29, 2013; U.S. patent application Ser. No. 20/090,189741 titled “WIRELESS SENSOR READER” having a publication date of Jul. 30, 2009; U.S. Pat. No. 8,382,677 titled “An anchored implantable pressure monitor” having an issue date of Feb. 26, 2013; U.S. Pat. No. 10,003,862 titled “Wireless sensor reader” having an issue date of Jun. 29, 2018; U.S. Pat. No. 9,894,425 titled “Wireless sensor reader” having an issue date of Feb. 13, 2018; U.S. Pat. No. 9,721,463 titled “Wireless sensor reader” having an issue date of Aug. 1, 2017; U.S. Pat. No. 9,489,831 titled “Wireless sensor reader” having an issue date of Nov. 8, 2016; US Patent Application 2022/0079456 titled “System and method for calculating a lumen pressure utilizing sensor calibration parameters” having a publication date of Mar. 17, 2022; U.S. Pat. No. 8,570,186 titled “Wireless sensor reader” having an issue date of Oct. 29, 2013; U.S. Pat. No. 8,493,187 titled “Wireless sensor reader” having an issue date of Jul. 23, 2013; U.S. Pat. No. 8,432,265 titled “Wireless sensor reader” having an issue date of Apr. 30, 2013; U.S. Pat. No. 8,154,389 titled “Wireless sensor reader” having an issue date of Apr. 10, 2012; U.S. patent application Ser. No. 20170309164 titled “WIRELESS SENSOR READER” having a publication date of Oct. 26, 2017; U.S. patent application Ser. No. 20/170,055048 titled “WIRELESS SENSOR READER” having a publication date of Feb. 23, 2017; U.S. patent application Ser. No. 20/160,210846 titled “WIRELESS SENSOR READER” having a publication date of Jul. 21, 2016; U.S. patent application Ser. No. 20/140,306807 titled “WIRELESS SENSOR READER” having an issue date of Oct. 16, 2014; U.S. patent application Ser. No. 20/140,028467 titled “WIRELESS SENSOR READER” having a publication date of Jan. 30, 2014; U.S. patent application Ser. No. 20/130,222153 titled “WIRELESS SENSOR READER” having a publication date of Aug. 29, 2013; U.S. patent application Ser. No. 20/090,189741 titled “WIRELESS SENSOR READER” having a publication date of Jul. 30, 2009; U.S. patent application Ser. No. 20/150,208929 titled “PRESSURE SENSOR, ANCHOR, DELIVERY” having a publication date of Jul. 30, 2015; U.S. patent application Ser. No. 20/090,009332 titled “SYSTEM AND METHOD FOR MONITORING INGESTED MEDICATION VIA RF WIRELESS TELEMETRY” having a publication date of Jan. 8, 2009; and U.S. Pat. No. 8,382,677 titled “An anchored implantable pressure monitor” having an issue date of Feb. 26, 2013. The patents, applications and publications listed herein are expressly incorporated by reference in their entireties.

Embodiments may be implemented in connection with concepts described in the following patents, all of which are expressly incorporated in their entireties by reference: U.S. patent application Ser. No. 20/210,369123 titled “Implantable wireless pressure sensor and method of manufacture” having a publication date of Dec. 2, 2021; U.S. Pat. No. 11,033,192, titled “Wireless Sensor for Measuring Pressure” having an issue date of Jun. 15, 2021; U.S. Pat. No. 9,610,444, titled “Erythropoeitin production by electrical stimulation” having an issue date of Apr. 4, 2017; U.S. Pat. No. 10,967,189, titled “Methods and systems for selectively delivering different types of bi-ventricular pacing” having an issue date of Apr. 6, 2021; U.S. patent application Ser. No. 20/180,303426, titled “Anchoring system for a catheter delivered device” having a publication date of Oct. 25, 2018; U.S. Pat. No. 10,638,955, titled “Pressure sensing implant” having an issue date of May 5, 2020; U.S. patent application Ser. No. 18/472,520, titled “Tandem Interlace Delivery Catheter for Delivering an Intracorporeal Sensor”, filed on Sep. 22, 2023; and U.S. Patent Application Ser. No. 63/427,122 titled “Sheathed Delivery Systems for Delivering an Intracorporeal Sensor”, filed on Nov. 22, 2022.

### Terms

The terms “implantable sensor” and “implantable wireless sensor” shall mean a sensor that is configured to be inserted and fixed within a body. The terms sensor, implantable sensor, and implantable wireless sensor are used interchangeably herein. For example, the sensor can be deployed to a deployment location within anatomy of the body using delivery systems such as a sheathed delivery system, a tethered delivery system, and/or a tandem delivery system. In some cases, the sensor can be a pressure sensor, while in other cases the sensor can detect other indications within the body. The sensor includes four side surfaces that are configured to face walls of the lumen and two end surfaces that are configured to face in proximal and distal directions. The four side surfaces can be referred to as top and/or bottom surface, first, second, third, and/or fourth surface. Opposite side surfaces are configured to face opposite walls of the lumen, and top and bottom surfaces are configured to face opposite sides of the lumen. It should be understood that a cross-sectional area of the sensor may be circular, square, oval, rectangular, irregular in shape, etc., and thus the description herein of “surface” etc., is not limited to flat or planar surfaces. The references to “top”, “bottom”, “front”, “back”, “side”, “walls”, “surface”, “first”, “second”, “third”, and “fourth” are for ease of discussion only and are not limiting.

The terms “lodge”, “lodging”, and “anchoring” shall mean securing anchoring element(s) of the sensor within lumen(s) to fix the sensor at a deployment location.

The term “anchoring element” shall mean any feature coupled to the sensor that is used to lodge the sensor within the lumen at the deployment location. The anchoring element is configured to engage walls of the lumen to provide rotational and axial stability of the sensor.

The term “attachment feature” shall mean any feature associated with, attached to, and/or integral/integrated with the sensor and/or anchoring element that can be used to removably couple the sensor and/or anchoring element to one or more components of one or more of the delivery systems.

The terms “distal end” and “proximal end” shall mean opposite ends of the sensor that face the proximal direction (e.g., toward a practitioner) and the distal direction (e.g., toward the deployment location), respectively. The distal end and proximal end, as discussed herein associated with the coupling of anchoring element(s), shall mean a portion of the sensor proximate opposite ends of the sensor and can include the end surfaces and/or the side surfaces.

The term “asymmetric”, when used to describe a relation between two or more anchoring elements shall mean a difference in a size (e.g., length, width), shape, orientation, and/or a number of elements of at least one anchoring element with respect to at least one other anchoring element at a time when the anchoring elements are freely released and not subject to any bias. For example, first and second anchoring elements are asymmetric, with respect to one another, when the first and second anchoring elements have different size, shape and/or orientation prior to being releasably attached or releasably retained in a fixed relationship with a delivery system and prior to being released within a lumen. Nonlimiting examples of asymmetric sensors are: i) anchoring element(s) coupled to one end of the sensor and no anchoring element coupled to the other end, ii) an anchoring element coupled to one end of the sensor that is larger in size than the anchoring element coupled to the other end, iii) anchoring elements configured to be oriented at non-zero angles with respect to one or more reference plane that differ from one end of the sensor to the other, iv) anchoring element(s) configured to be shaped to extend through multiple parallel planes, and v) an anchoring element having a single element coupled to one end of the sensor and an anchoring element having more than one element coupled to the other end of the sensor. Two or more anchoring elements are “geometrically” asymmetric with respect to one another when the first anchoring element has a different size, shape and/or orientation than the second anchoring element. Two or more anchoring elements are “numerically” asymmetric with respect to one another when the first anchoring element has a first number of elements (e.g., one loop) and the second anchoring element has a different number of anchoring elements (e.g., two or more loops).

The terms “sheathed delivery system” and “sheath delivery system” shall mean a system that includes at least one hollow tube or sheath (e.g., delivery sheath) that delivers a sensor to a position within a lumen. The sensor can be retained or contained within the sheath, confining the one or more anchor elements in an undeployed state to minimize or prevent contact/entanglement with anatomy. The sensor is configured with a release element (e.g., threaded push cable, push snare, suture) that allows passage of the sensor through the tube. The sensor is advanced out of the tube at the desired location, released from the release element, and the one or more anchor elements expand to anchor the sensor within the lumen.

The terms “push rod” and “push catheter” shall mean any wire, rod, tube, and/or assembly of components such as rod(s), sheaths, and the like that push a sensor within a sheath (e.g., delivery sheath) to a delivery location within a body. In some embodiments, the push catheter can be a tether, twisted multistrand cable, a composite layered cable such as polymer around a wire, braided polyimide, peek extrusion, hypotube, solid wire, etc., that can releasably retain an anchoring element of the sensor.

The terms “tethering delivery system”, “tethered delivery system”, and “tether delivery system” shall mean a system that pseudo-sews the sensor onto a shaft of the delivery system, such as with a tether, thread, wire, floss, etc.

The tether is configured to be a release element that removably couples one or more components integrated with the sensor to the delivery system.

The term “skive” shall mean an area of removed catheter material, such as an opening or notch in a catheter that is accessible from outside the catheter. The skive exposes a lumen within the catheter and can create an opening for a component (e.g., a portion of an anchor loop, tether, thread, floss, etc.) to pass through and/or be secured.

The term “tandem delivery system” shall mean a system having a tandem configuration wherein a pressure sleeve can be proximal and coaxial to the sensor which is mounted on a rail. The term “tandem” shall mean one behind another and/or end to end in a locked manner. For example, a sensor and a delivery catheter can be held in tandem by locking the sensor to a “rail”, such as a guidewire lumen that extends through the delivery catheter. The guidewire lumen is configured to be a release element that removably couples one or more components integrated with the sensor to the delivery system.

The terms “releasably attached”, “releasably retained”, “releasably retaining”, and “releasably retain” shall mean to hold in a fixed relationship, such as with a delivery system, while allowing the release at a desired position. The terms may be used interchangeably herein.

The terms “center” and “centered” shall mean that the sensor is located in a central position with respect to a diameter of a lumen, roughly halfway from opposite walls of the lumen.

The terms “off-center” and “off-centered” shall mean that the sensor is located in a position with respect to a diameter of a lumen that is closer to one wall of the lumen than it is to an opposite wall of the lumen.

The terms “alter shape”, “altering shape”, and “change shape” are used interchangeably herein and shall mean that one or more of a diameter, length, overall shape, and/or shape features such as flat edges, rounded edges, rounded corners, and anchor points of an anchoring element can be changed, modified or adjusted as the result of pressure outside of the anchoring element and/or movement of the anchoring element within a lumen. For example, the shape of an anchoring element can be altered to reform the shape in a more positive, interactive way with respect to the lumen. The anchoring element can be altered to i) improve the contact/pressure between the anchoring element and lumen to resist further movement, and/or ii) facilitate movement in a desired direction.

### Overview

When a sensor is implanted within a body, it is important to have enough force or friction between the anchor loop(s) and the anatomy to prevent the sensor from moving from its desired location. In some anatomy, such as pulmonary arteries, the branches taper more quickly. With sensors that have anchoring elements that are the same size at either end, the distal anchor loop may accomplish most of the anchoring functionality while the proximal anchor loop may not fully engage the anatomy. In other anatomy such as peripheral vascular vessels, the vessels or lumens get larger toward the heart and smaller away from the heart, and thus the proximal anchor loop may provide most of the anchoring functionality. In other cases, sensors may mount an integrated sensing pad on various surfaces of the sensor. It is desirable to position a sensor pad centrally within the lumen, and thus the sensor may be centered or off-centered within the cross-sectional diameter of the lumen to improve the data collected by the sensor.

In accordance with new and unique aspects herein, sensors are described that can have different sized proximal and distal loops to fully engage lumens with different diameters. Further, sensors can have multiple elements at the distal end that are oriented in different directions to engage multiple different branches, or smaller lumens, than the anchoring element at the proximal end.

Sensors herein provide improvements and practical applications to improve the placement and reduce the migration of sensors within the body. Sensors can have anchoring elements with different shaped loops, fingers, etc., and some may include anchoring elements only on one end to suit a particular anatomical application.

Sensors herein provide improvements in interfacing with delivery systems, such as attachment features on anchoring elements. Each of the sensors herein are configured to be releasably attached to or releasably retained by a delivery system. Further, anchoring elements described herein can alter shape, such as in diameter, length, and/or overall shape (e.g., more or less pointed, more or less curved, more or less straight or flat edged), when moved within the lumen to either facilitate placement within a more narrow lumen, or to resist migration in a particular direction. In some embodiments, sensors can be configured to interface with both tethered delivery systems and tandem delivery systems. In other embodiments, when used with sheathed delivery systems, sensors can be configured to be re-capturable or re-sheathable by withdrawing the sensor or portions of the sensor, such as one or more of the anchoring elements, into the delivery sheath after the sensor has been deployed in the lumen. In this example, the anchoring elements can be re-deployed (e.g., advanced out of the delivery sheath) multiple times (e.g., up to five times) while retaining consistent deployed shape and anchoring capability/functionality. This provides the advantage of moving the sensor to a different location and/or orientation if needed.

Further improvements herein are the integration of one or more radiopaque elements on the body of the anchoring element. Radiopaque elements can be positioned such that physicians can visualize, such as during fluoroscopy, the anchoring element during placement or removal of the sensor.

### Implantable Sensors Having Asymmetric Features Associated With Anchoring Elements

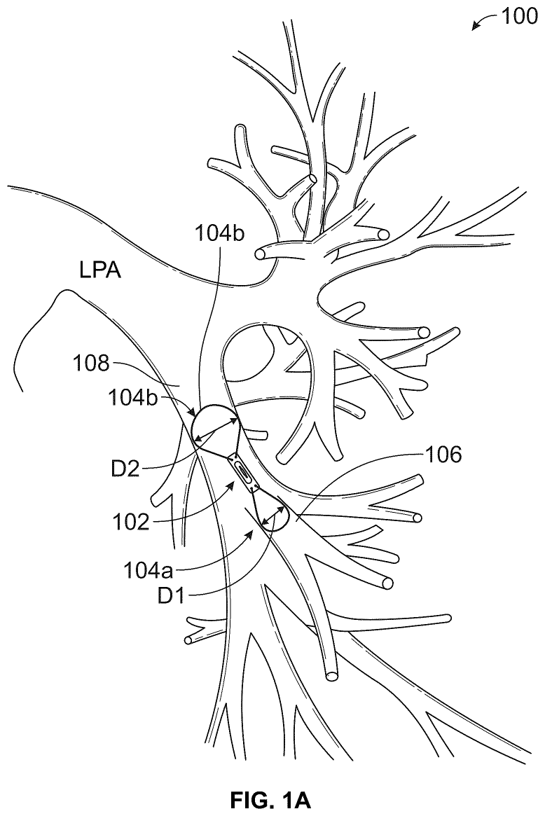

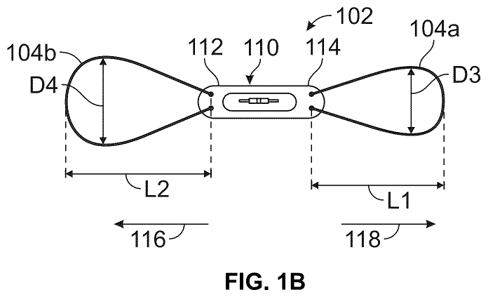

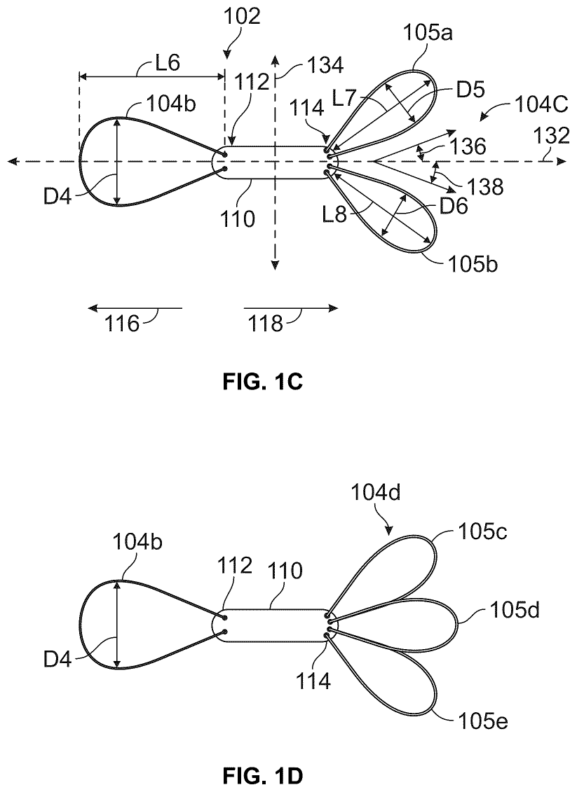

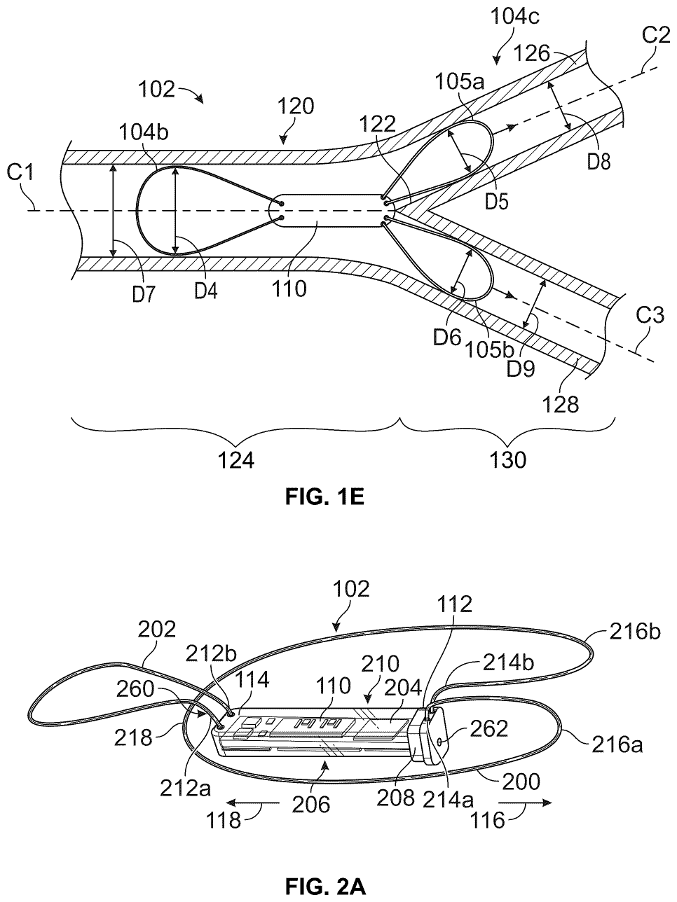

FIG. 1A illustrates an example of pulmonary branches 100 having a sensor 102 with asymmetric anchoring elements lodged therein in accordance with embodiments herein. The pulmonary branches 100 are those of the left pulmonary artery (LPA); however, it should be understood that the sensor 102 can be anchored within one or more lumens of any appropriate organ or vessel, such as an artery, an arteriole in the pulmonary artery vasculature, right side pulmonaries, renal and/or other vasculature. An anchoring element 104a is coupled to a distal end of the sensor 102 and an anchoring element 104b is coupled to a proximal end of the sensor 102.

Diameter D1 indicates a diameter of lumen 106, which is smaller than diameter D2 of lumen 108. The anchoring element 104a is relatively smaller than the anchoring element 104b when released without bias, such as prior to implant, and spans the diameter D1 of lumen 106 to securely lodge the anchoring element 104a within the lumen 106. The anchoring element 104b is relatively larger than the anchoring element 104a, and spans the diameter D2 of lumen 108 to securely lodge the anchoring element 104b within the lumen 108.

FIG. 1B illustrates a top view showing the sensor 102 with anchoring elements 104a, 104b of FIG. 1A in accordance with embodiments herein. The sensor 102 has a sensor body 110 having a proximal end 112 and a distal end 114. For reference, proximal direction 116 and distal direction 118 are indicated. For example, proximal direction 116 or orientation indicates the portion of the sensor 102 closest to the practitioner.

The anchoring element 104a coupled to the distal end 114 of the sensor body 110 has a diameter D3 and the anchoring element 104b coupled to the proximal end 112 of the sensor body 110 has a diameter D4. The diameters D3 and D4 can be associated with the widest portion of the anchoring elements 104a and 104b, respectively, prior to being deployed. Prior to or in advance of being deployed within a lumen, the diameter D3 of the anchoring element 104a is less than the diameter D4 of the anchoring element 104b.

Similarly, the anchoring element 104a has a length L1 and the anchoring element 104b has a length L2. The length L1 can be less than the length L2. In other embodiments, the length L1 can be greater than the length L2.

The anchoring elements 104a, 104b can be used to attach the sensor 102 to a delivery system, arranged/captured within a delivery sheath, and the like, prior to deploying the sensor 102 within the lumen. The anchoring elements 104a, 104b can be formed of nitinol or other shape memory material as is known in the art. Therefore, the anchoring elements 104a, 104b can be compressed to different shapes when integrated with a delivery system, and will deploy to their preconfigured shape when delivered into the lumen.

It should be understood that in some embodiments the configuration of the features of the distal and proximal anchoring elements 104 can be swapped. For example, referring to FIG. 1B, the distal anchoring element 104a may instead have a larger diameter than the proximal anchoring element 104b. This can be advantageous if the sensor 102 is used in peripheral vascular areas, for example.

Each of the anchoring elements 104 of the sensors 102 described herein has a shape, size (e.g., length, width), and orientation with respect to the sensor body 110 and/or each other prior to implant within the lumen. The shape, size, and orientation can be achieved by using shape memory material such as but not limited to nitinol, MP35, Eligiloy, shape memory polymers such as Nylon, etc., barium filled polymer, bioabsorbable materials and/or polymers such as poly lactic-co-glycolic acid (PLGA), L-lactide (LLA), polylactic acid (PLA), etc., by selection of the surface and location where each of the anchoring elements 104 are coupled to the sensor body, and the like. The length shall be a distance from the closest surface of the sensor body 110 to an outermost end surface or farthest position of the anchoring element 104. A width shall be a widest distance across the anchoring element 104. The lengths and widths may or may not be located at the corresponding central locations of the anchoring element 104 depending upon the shape of the element 104. The shape, size, and orientation of each of the anchoring elements 104 can be determined in advance of deployment or when the anchoring elements 104 are released without bias, and/or prior to interconnection with a delivery system, which compresses or otherwise alters or changes the shape of the anchoring elements 104 while securely retaining the sensor 102 to/within the delivery system. It is understood that when the anchoring element 104 is deployed within a lumen, the anchoring element 104 may not have the same shape, length, width, and orientation (compared to being released without bias) based on the size (e.g., diameter) and orientation of anatomy.

FIGS. 1C and 1D illustrate top views of the sensor 102 having the anchoring element 104b coupled to the proximal end 112 of the sensor body 110 and an anchoring element 104c that includes a plurality of elements 105 coupled to the distal end 114 in accordance with embodiments herein. Turning first to FIG. 1C, elements 105a, 105b of the anchoring element 104c are coupled to the distal end 114. The three anchoring elements 104b, 105a, 105b can provide anchoring within three different lumens or vessels.

The element 105a has a diameter D5 and the element 105b has a diameter D6. The diameters D5 and D6 can be substantially similar or different with respect to each other. The diameters D5 and D6 can be determined based on diameter(s) of targeted anatomy. The diameter D4 of the anchoring element 104b can be larger than one or both of the diameters D5 and D6.

The length L6 of the anchoring element 104b, length L7 of element 105a, and length L8 of element 105b can be the same or different with respect to each other. In some embodiments, the lengths L7 and L8 can be shorter than the length L6.

Further, the orientation of the elements 105a and 105b can be determined to project at predetermined angles or in directions off a central axis with respect to the sensor body 110 based on targeted anatomy. The central axis extends along a length of the sensor body 110 and may also be referred to as a longitudinal axis. For example, a horizontal plane 132 extends along the sensor body 110 in the proximal and distal directions 116, 118 and a vertical plane 134 extends perpendicular to the horizontal plane 132 and the longitudinal axis. Each of the anchoring elements 104 and elements 105 can project at angles away from one or both of the horizontal and vertical planes 132, 134.

In some embodiments, the anchoring element 104b has the length L6 and is coupled to the sensor body 110 to extend at a first orientation to the sensor body 110, such as to extend in the proximal direction 116 at approximately zero degrees with respect to the horizontal plane 132 and the vertical plane 134. The anchoring element 104b is configured to expand to the loop shape as shown, or other shape defined by shape memory material, up to the diameter D4. The element 105a has the length L7 and is coupled to the sensor body 110 to extend at a second orientation to the sensor body 110, such as to extend at a non-zero angle 136 with respect to the horizontal plane 132. The element 105a may extend at approximately zero degrees with respect to the vertical plane 134. The element 105b has the length L8 and is coupled to the sensor body 110 to extend at a third orientation to the sensor body 110, such as to extend at a non-zero angle 138 with respect to the horizontal plane 132. The element 105b may extend at approximately zero degrees with respect to the vertical plane 134. In some embodiments, the anchoring element 104 is configured to extend through multiple planes, such as at least partially horizontally and it least partially vertically.

Although the angles 136, 138 are illustrated as acute with respect to the horizontal plane 132, the angles 136, 138 can be 90 degrees with respect to the horizontal plane 132, or can be obtuse angles. Anchoring elements 104 having obtuse angles can be configured to face generally in the opposite direction. For example, in some embodiments one of the elements 105 can be configured with an obtuse angle to generally face in the proximal direction 116. Similarly, each of the anchoring elements 104 can be configured to have non-zero angles with respect to the vertical plane 134.

Turning to FIG. 1D, anchoring element 104d includes elements 105c, 105d, 105e coupled to the distal end 114 of the sensor body 110. The elements 105c, 105d, 105e can be positioned close together when deployed, fanned in different directions (e.g., angles) relative to a central axis (e.g., longitudinal axis) with respect to the sensor body 110, and have different or the same diameters and lengths. For example, the element 105d can project substantially along a central axis with respect to the sensor body 110, while the elements 105c, 105e project outwardly with respect to the central axis. In other cases, all of the elements 105c, 105d, 105e can project outwardly with respect to the central axis. Similar to FIG. 1C, the diameters of the elements 105c, 105d, 105e can be substantially similar or different with respect to each other, determined based on targeted anatomy, and the diameter D4 of the anchoring element 104b can be larger than one, two, or all of the diameters of the elements 105c, 105d, 105e. It should be understood that there can be more than three elements 105 coupled to the distal end 114 of the sensor body 110.

FIG. 1E shows the sensor 102 of FIG. 1C having the anchoring elements 105a, 105b lodged within a lumen 120 proximate to where the lumen 120 bifurcates at point 122 in accordance with embodiments herein. As shown, the lumen 120 includes first and second branches 126, 128.

The central axis of the sensor body 110 within the lumen 120 is indicated with dotted line C1. In the embodiment shown, the anchoring element 104b is oriented in line with the central axis C1 (e.g., angular orientation of zero degrees). Orientations of the first and second branches 126, 128 of the lumen 120 are indicated with dotted lines C2, C3, respectively. The lines C2, C3 extend at angles away from the line C1.

The lumen 120 has a single lumen portion 124 having a diameter D7. The diameter D4 can be determined such that when the sensor 102 is deployed within the single lumen portion 124, the anchoring element 104b engages the walls of the single lumen portion 124 with enough force and/or friction to lodge the sensor 102 within the lumen 120. The diameters D5 and D6 of the elements 105a, 105b can be determined such that when the sensor 102 is deployed within the lumen 120, the element 105a engages the walls of the first branch 126 of bifurcating portion 130 and the element 105b engages the walls of the second branch 128 of bifurcating portion 130 with enough force and/or friction to securely lodge the sensor 102 in the lumen 120.

In accordance with new and unique aspects, embodiments of the sensor 102 advantageously provide the ability to position the sensor 102 in a desired position. For example, the sensor 102 can be positioned proximate where a lumen splits into more than one lumen. In other cases, one or more small vessels/lumens can be used to position the sensor 102 superior of where the lumen is wider. Further, providing anchoring elements 104, 105 of different sizes, numbers, shapes, and/or orientation with respect to the central axis of the sensor body 110 improves the retention of the sensor 102 in the desired location.

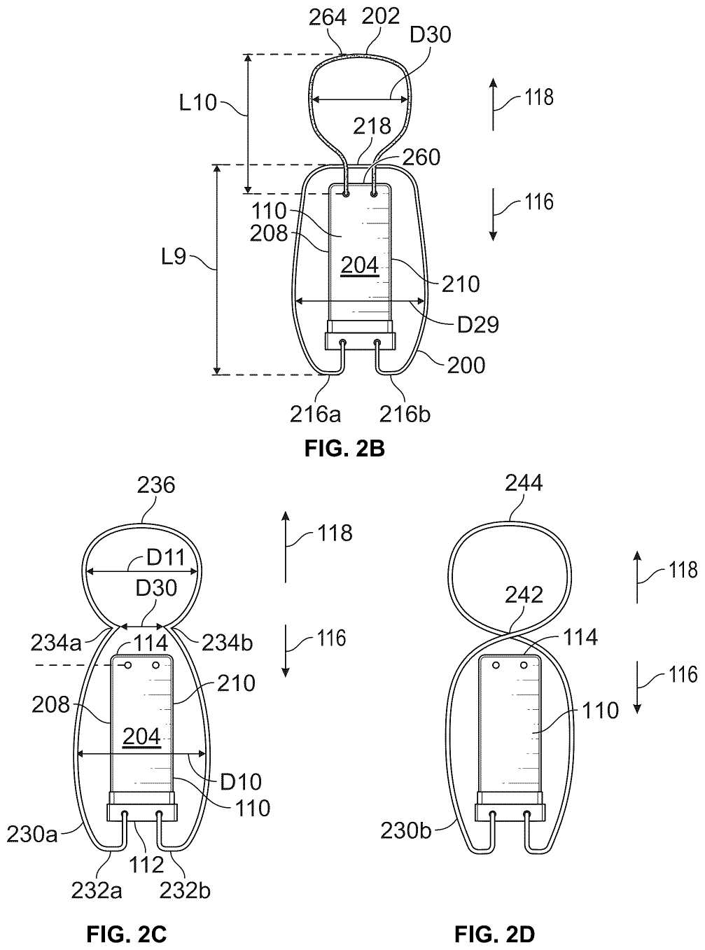

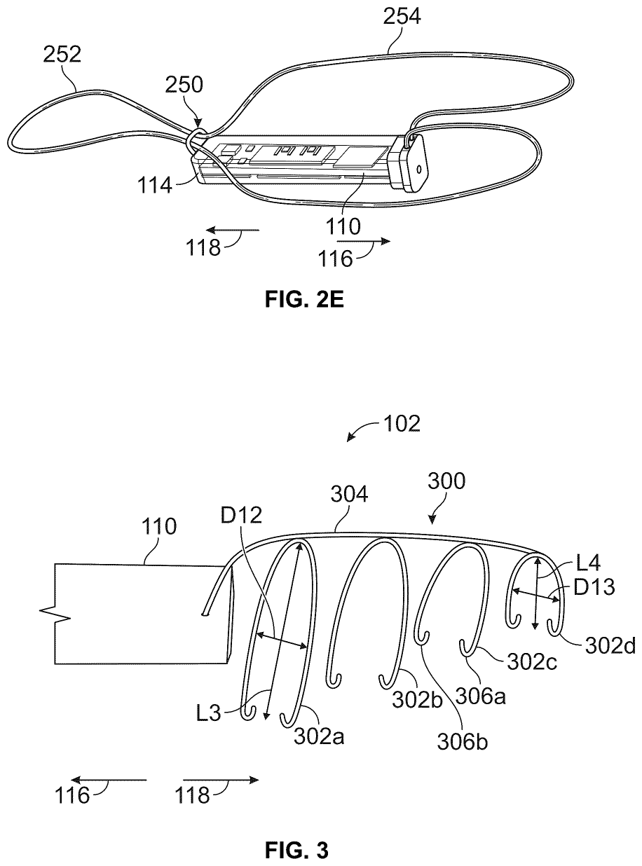

FIG. 2A is an isometric view of a sensor 102 having anchoring elements coupled to the proximal and distal ends 112, 114 of the sensor body 110 that both project in the distal direction 118 in accordance with embodiments herein. FIG. 2B shows a top view of the configuration of FIG. 2A. One of the anchoring elements is a body anchoring loop 200 that is coupled the proximal end 112 of the sensor body 110 and another of the anchoring elements is a distal anchoring loop 202 that is coupled to the distal end 114 of the sensor body 110. In other embodiments, the anchoring elements can both project in the proximal direction 116, such that the body anchoring loop 200 is coupled to the distal end 114 of the sensor body 110 and the anchoring loop 202 can be a proximal anchoring loop that is coupled to the proximal end 112 of the sensor body 110. An advantage of the anchoring element(s) discussed in FIGS. 2A-2E is that the anchoring element is re-capturable with some delivery systems, such as sheathed delivery systems. The anchoring element (and in some cases the sensor 110) can be pulled back into a sheath, such as by the release element or other element of the delivery system, then readvanced and deployed to anchor as planned in another location.

The sensor body 110 can have a top surface 204, bottom surface 206, first side surface 208, and second side surface 210 (not visible in FIG. 2A) that extend between the distal and proximal ends 112, 114. Legs 212a, 212b of the distal anchoring loop 202 extend upwardly out of the top surface 204, and the distal anchoring loop 202 is bent to extend coplanar with the distal direction 118. Legs 214a, 214b of the body anchoring loop 200 extend upwardly out of the top surface 204, and the body anchoring loop 200 is bent to extend coplanar with the proximal direction 116. The body anchoring loop 200 is then bent to form proximal ends 216a, 216b and extends in the distal direction 118 along the first and second side surfaces 208, 210 and projects beyond the distal end 114 of the sensor body 110. A distal end 218 of the body anchoring loop 200 extends and is positioned under the distal anchoring loop 202.

In other embodiments, the legs 212a, 212b can project in the distal direction 118, such as by protruding or extending from a distal end surface 260 of the sensor body 110 at the distal end 114. In still other embodiments, the legs 214a, 214b can project in the proximal direction 116, such as by protruding from a proximal end surface 262 of the sensor body 110 at the proximal end 112.

At least portions of the body anchoring loop 200 and the distal anchoring loop 202 are configured to engage walls of the lumen (not shown) to lodge the sensor 102 in the lumen.

Referring to FIG. 2B, when the sensor 102 is not implanted and no force is placed on the body anchoring loop 200 or the distal anchoring loop 202, a length L9 of the body anchoring loop 200 can extend from proximal end 216 to distal end 218. A length L10 of the distal anchoring loop 202 can extend from an end surface 260 of the sensor body 110 to a farthest position 264 (e.g., far distal end point) of the distal anchoring loop 202. In some embodiments, the length L9 of the body anchoring loop 200 can be greater than the length L10 of the distal anchoring loop. In some embodiments, a diameter D29 of the body anchoring loop 200 can be greater than a diameter D30 of the distal anchoring loop 202. This can provide an advantage when implanted in an artery wherein the distal vasculature narrows. Therefore, the distal anchoring loop 202 can securely anchor the sensor 110 in more narrow vasculature, while the body anchoring loop 200 can, at the same time, securely anchor in wider vasculature.

FIGS. 2C and 2D show top views of a single anchoring element 230 in accordance with embodiments herein. Turning first to FIG. 2C, the anchoring element 230a is coupled to the proximal end 112 of the sensor body 110. As with the body anchoring loop 200 of FIGS. 2A and 2B, the anchoring element 230 has two legs that extend upwardly out of the top surface 204 (as shown in FIG. 2A) and is bent to extend in the proximal direction 116. The length of the bend above the sensor 102 can control how the sensor 102 sits in the lumen. For example, if the sensor 102 is relatively small and the lumen is relatively large with respect to each other, the length of the leg before the bend may be long to assure that the sensor 102 is positioned in contact with the wall of the lumen, if desired. In other cases, if the sensor 102 and lumen are closer in size with respect to each other, the length of the leg before the bend may be relatively shorter. The anchoring element 230a is then bent to form proximal ends 232a, 232b and to extend generally in the distal direction 118. The anchoring element 230a extends along the sensor body 110, such as along the first and second side surfaces 208, 210 and projects beyond the distal end 114 of the sensor body 110, which is the opposite end to which the anchoring element 230a is attached. It should be understood that the proximal ends and distal ends of the anchoring elements can be rounded, form a point, be substantially squared off, etc., and that the bends of the anchoring elements as discussed herein are not limited to certain shapes.

The anchoring element 230 can have a tortuous shape (e.g., marked by bends, twists, turns, curves, cross-overs). For example, the anchoring element 230a can be bent on either side of the loop, such as beyond the distal end 114 of the sensor body 110. The anchoring element 230a can be bent to project inwardly, away from the lumen, such as at bends 234a, 234b. The anchoring element 230a can be curved to form a curved loop 236. At least a portion of the anchoring element 230a, such as portions extending along the first and second side surfaces 208, 210 and side portions that extend distally of the first and second inward bends 234a, 234b, is configured to engage the lumen walls.

The anchoring element 230a can have a diameter D10 associated with a central portion of the sensor body 110, a diameter D11 associated with a distal portion of the anchoring element 230a, and a diameter D30 associated with a distance between the inward bends 234a, 234b. It should be understood that the diameters D10 and D11 and the discussion can apply equally to the embodiments shown in FIGS. 2A, 2B, and 2D. The diameters D10 and D11 can be substantially the same or different with respect to each other. In some embodiments, it may be advantageous to make both the diameters D10 and D11 approximately the same, which in other embodiments, it may be advantageous to make the diameters D10 and D11 different to accommodate different diameters of the lumen. The diameter D30 can be less than the diameters D10 and D11.

Turning to FIG. 2D, anchoring element 230b can be attached to the sensor body 110 as discussed in FIG. 2C. The anchoring element 230b extends beyond the distal end 114 of the sensor body 110 and one side of the loop is crossed over the other side of the loop at cross-over 242 (e.g., in a figure eight shape), forming a distal loop 244 that is integral with the anchoring element 230b. In other embodiments, the anchoring element 230b can include a full twist.

For example, if the distal loop 236, 244 is in a smaller diameter lumen than the body loop, the distal loop 236, 244 squeezes smaller and the anchoring element 230 will expand the proximal portion or body loop to be larger. Similarly, if the diameter of the lumen associated with the distal loop 236, 244 is larger, the proximal portion or body loop squeezes smaller and the anchoring element 230 will expand the distal loop 236, 244. This flexibility in the diameters of the proximal/body and distal loops can improve anchoring within lumens that have varying diameters, such as by allowing tissue to conform around the different sections to provide multiple anchor points/options to prevent the sensor 110 from migrating.

FIG. 2E illustrates a restraining feature that restrains the anchoring element(s) in accordance with embodiments herein. The anchoring element(s) can be either two loops, such as the body anchoring loop 200 and distal anchoring loop 202 of FIGS. 2A and 2B, or can be a one-piece loop, such as the anchoring element 230a of FIG. 2C or the anchoring element 230b of FIG. 2D. A restraining feature 250 is coupled to the distal end 114 of the sensor body 110.

If a single loop is used, the restraining feature 250 controls the two loop sections (e.g., body anchoring loop 254 and distal anchoring loop 252) together. The restraining feature 250 can prevent excessive movement of the body anchoring loop 254 in the distal direction 118 if the body anchoring loop 200 is squeezed. Without the restraining feature 250, the two loop sections can shift with respect to each other. The restraining feature 250 provides a focal point that can exaggerate the squeeze and expansion effect.

If a single anchoring loop is used, the restraining feature 250 holds the anchoring element while allowing some movement. The anchoring element may be formed similar to the anchoring elements 230a or 230b, having bends 234 (see FIG. 2C), a cross over 242 (see FIG. 2D), etc., positioned at the desired location with respect to where the restraining feature 250 is located proximate the distal end 114. In this manner, a distal loop 252 and a body loop 254 are formed from the single loop. If the distal loop 252 gets squeezed (e.g., receives squeezing pressure such as from being moved or migrating in the distal direction 118 into a relatively smaller lumen), the single loop alters shape and portions of the distal loop 252 move in the proximal direction 116 to expand the body loop 254. If the body loop 254 gets squeezed (e.g., receives squeezing pressure such as from being moved or migrating in the proximal direction 118 into a relatively smaller lumen), the single loop alters shape and portions of the body loop 254 move in the distal direction 118 to expand the distal loop 252. Accordingly, diameters of the distal and body loops 252, 254 can adjust dynamically to better lodge the sensor 102 in the lumen. For example, the diameter(s) of the distal and/or body loops 252, 254 may be increased to increase a length of surface area of the distal and/or body loops 252, 254 that are in direct contact with a surface of a lumen. Additionally or alternatively, the diameter(s) of the distal and/or body loops 252, 254 may be increased to increase an amount of outward force applied by the distal and/or body loops 252, 254 to the surface of a lumen. In particular, migration of the sensor 102 in the proximal direction 116 is prevented or minimized.

In some cases, it may be advantageous to select how the sensor 102 is positioned within the lumen, such as along a central portion of the lumen or adjacent a wall of the lumen. Referring to FIG. 2A-2E, in some embodiments, the body anchoring loop 200, anchoring element 230, and/or anchoring element 230 can be formed to extend along one side of the sensor body 110, such as to position the sensor body 110 proximate to or in communication with the lumen wall.

A disadvantage of current sensors that have the same size and orientation of the proximal and distal loops and that are delivered using a sheathed delivery system is that if the sensor is retracted back into the sheath for repositioning after the sensor was advanced beyond the sleeve, the proximal loop (e.g., closest to the practitioner) may contort into a shape or flip distally over the sensor body. This may result in unpredictability of the shape or placement of the proximal loop when the sensor is re-advanced out of the sheath for a second time for placement. This could interfere with readings of the sensor or the loop may possibility be in a shape that can cause more trauma than desired to the patient. In some cases, the sensor may not be usable and needs to be removed from the patient.

In some embodiments, the sensors 102 shown in FIGS. 2A-2E can be delivered into the body using a sheathed delivery system. In accordance with new and unique aspects, the sensors shown in FIGS. 2A-2E can be moved in and out of the delivery sheath multiple times (e.g., re-capturable up to five times) and the body anchoring loop 200 and distal anchoring loop 202, or the anchoring element 230a, 230b if used as a singular anchoring element, will consistently retain their shape when deployed, providing consistent anchoring within the lumen.

FIG. 3 illustrates a sensor 102 having an anchoring element 300 including a plurality of open-sided curved fingers 302 attached to a spline 304 in accordance with embodiments herein. The curved fingers 302 can be shaped like arches or horseshoes, for example, and can be formed of shape memory material(s) as discussed herein. Although four curved fingers 302a, 302b, 302c, 302d are shown, it should be understood that more or less fingers 302 can be used. The fingers 302 are shown having different sizes, such that a length L3 and diameter D12 of the finger 302a are greater than each of the other fingers 302b, 302c, 302d. The size of the fingers 302 may be tapered such that the lengths and diameters gradually decrease, and the outermost finger 302d has the smallest length L4 and diameter D13. This can be advantageous when the lumen is narrowing in diameter in the distal direction 118. In some cases, some of the fingers 302 may have the same diameter and width, and in other cases, all of the fingers 302 can have the same diameter and width.

Outer ends 306a, 306b (only indicated on the finger 302c) can be bent, have a blunt end to prevent puncture, include a welded ball, a ball of nitinol (or other shape memory material), be laser cut, have a polished eyelet or round feature, and the like. The spline 304 is stiff or strong enough to keep the fingers 302 from flipping back on each other as they are moved through the sheathed delivery system.

Anchoring Element that Alters Shape to Interface with Sheathed Delivery System

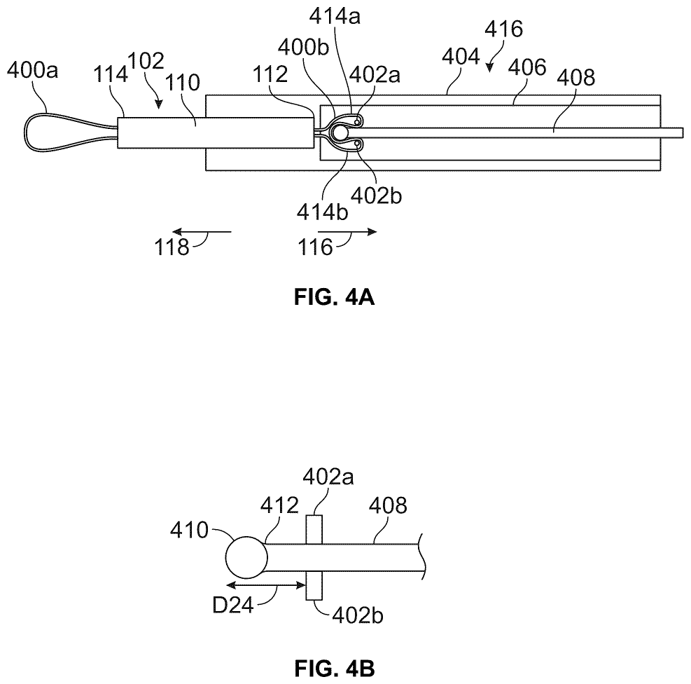

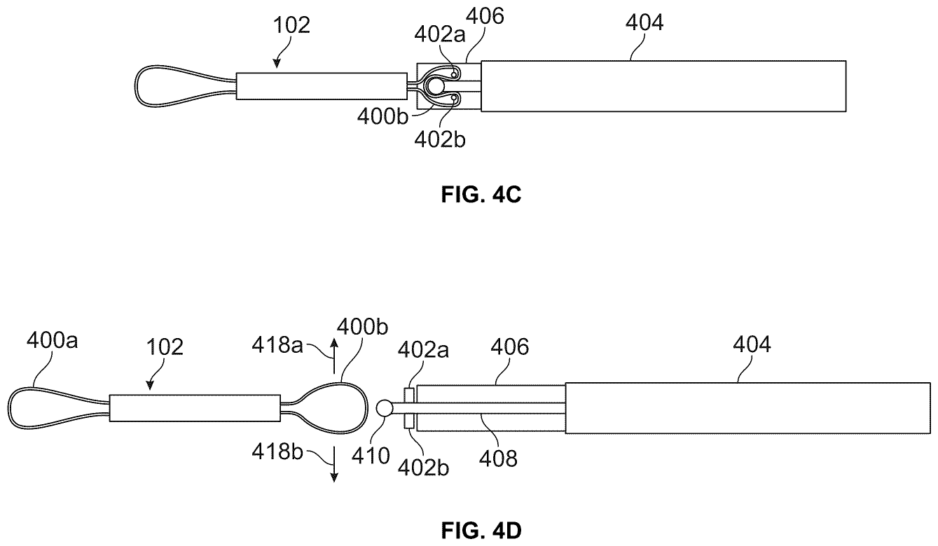

FIG. 4A shows a cross-sectional view of a sensor 102 that has an anchoring element 400b that folds in on itself when interconnected with a sheathed delivery system 416 in accordance with embodiments herein. An anchoring element 400a is coupled to the distal end 114 of the sensor body 110 and an anchoring element 400b is coupled to the proximal end 112. As shown, both of the anchoring elements 400a, 400b are loops, but the anchoring element 400a could have a different shape and/or size than the anchoring element 400b. Further, when released from the delivery system, the anchoring element 400b can retain a less folded shape, or return to a different shape that is not a uniform loop, such as substantially triangular, having more than two points configured to engage with the lumen walls, etc.

The sheathed delivery system 416 includes an outer sheath 404 and an inner sheath 406. As shown, the sensor 102 has been partially advanced out of the outer sheath 404.

FIG. 4B shows a cross-sectional view of the push rod 408 and interlocking feature in accordance with embodiments herein. The sheathed delivery system 416 includes a push rod 408 that interfaces with the anchoring element 400b. The push rod 408 can have a ball 410 on distal end 412, or the distal end 412 can be rounded, flat, etc. The ball 410 is pushed into the proximal end of the anchoring element 400b. The shape of the anchoring element 400b is altered into two half loops 414a, 414b, which loop over and are retained by protrusions or locking pins 402a, 402b, respectively. In other embodiments, a single locking pin can be used. A distance D24 between the ball 410 and the locking pins 402 along the push rod 408 is determined to hold the half loops 414 securely interfaced with the locking pins 402 so that the assembly can be mounted within the inner sheath 406, while also allowing the half loops 414 to rise off the locking pins 402 when the assembly is removed from the inner sheath 406. Although not shown, additionally or alternatively a relatively larger ball 410 can be pushed into the proximal end of the anchoring element 400b, altering the shape of the anchoring element 400b into, for example, two half loops without the use of a locking pin. The assembly can be positioned within the outer sheath 404 such as with a pressure fit without the locking pins 402a, 402b and/or inner sheath 406.

FIG. 4C shows a cross-sectional view wherein a distal portion of the inner sheath 406 has been advanced out of the outer sheath 404. The anchoring element 400b is still retained by the locking pins 402.

Turning to FIG. 4D, this figure shows a cross-sectional view wherein the sensor 102 has been fully advanced out of the inner sheath 406 (e.g., release element). The half loops 414a, 414b expanded outwardly in directions 418a, 418b, respectively, away from the push rod 408. The half loops 414a, 414b lifted off and away from the locking pins 402a, 402b, respectively, and the anchoring element 400b returned to its original, single loop shape. The anchoring elements 400a, 400b engage walls of the lumen (not shown) to lodge the sensor 102 within the lumen.

Sensors with Anchoring Elements Extending Horizontally and/or Vertically

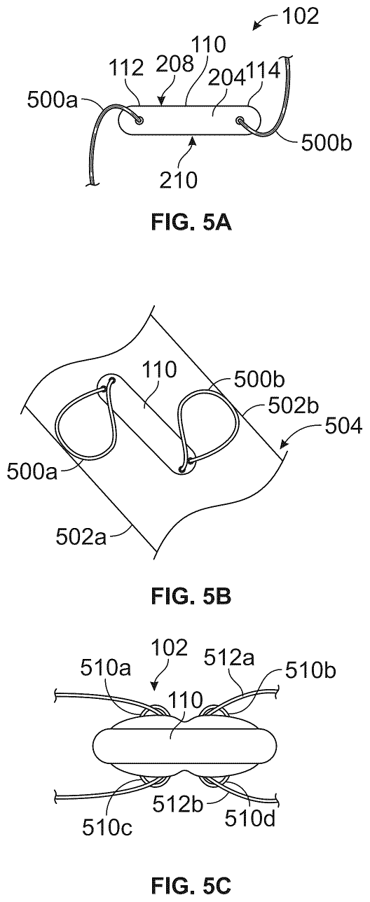

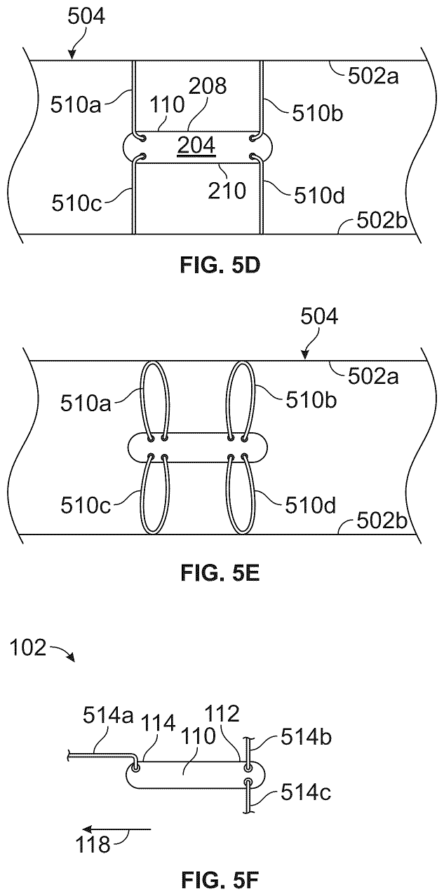

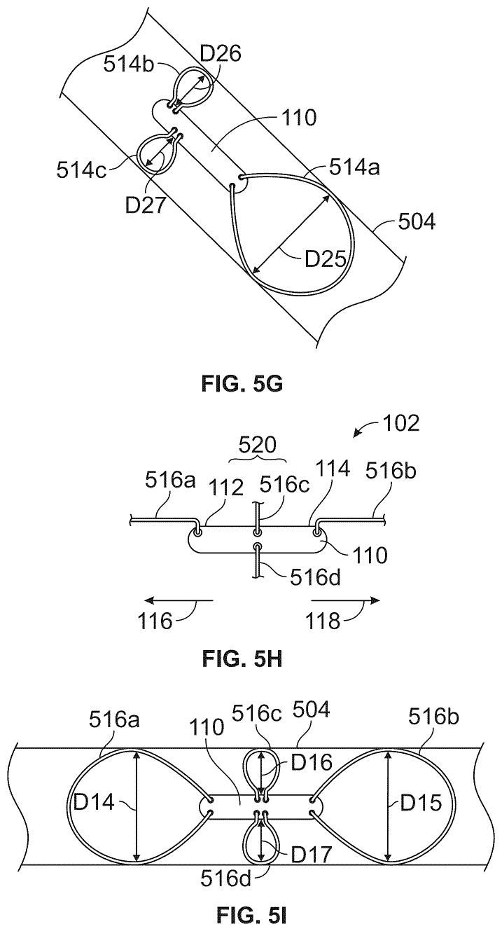

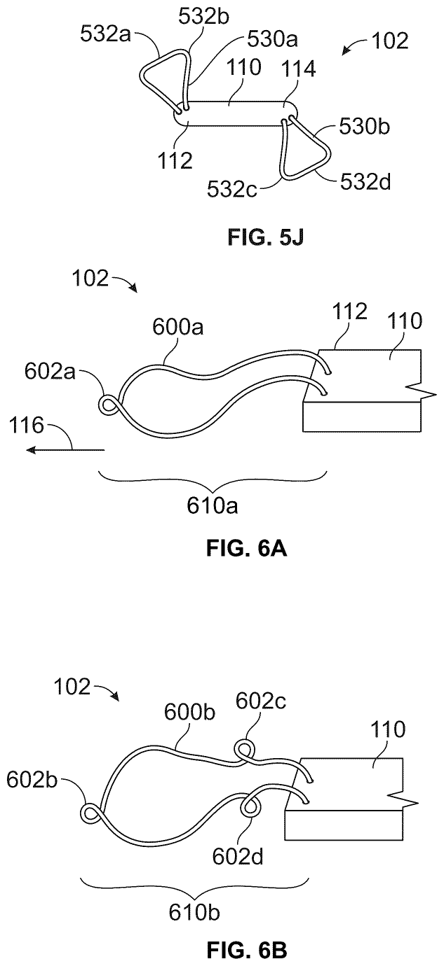

Turning to FIGS. 5A-5J, these figures show embodiments wherein at least some of the anchoring elements are configured to have an angular orientation to extend approximately vertically with respect to the sensor body 110 or horizontally (e.g., extending within the plane that is co-planar between the proximal direction 116 and the distal direction 118 and co-planar with the sensor body 110). The sensors 102 of FIGS. 5A-5J can be delivered into the lumen using either a sheathed delivery system or a tethering delivery system. It should be understood that the anchoring elements can be attached to the same surface of the sensor body 110 or to two or more surfaces of the sensor body 110.

FIG. 5A is a side view of the sensor body 110 and anchoring elements 500a, 500b that are configured to engage opposite sides of the lumen to position the sensor body 110 within the lumen in accordance with embodiments herein. The sensor body 110 is substantially rectangular in shape, and includes a top surface 204 that is opposite a bottom surface (not shown), and a first side surface 208 that is opposite a second side surface 210.

The anchoring elements 500a, 500b are coupled to the top surface 204 proximate proximal and distal ends 112, 114, respectively. It should be understood that the anchoring elements 500a, 500b can be coupled to the bottom surface (not shown), the first or second side surfaces 208, 210, end surfaces of the sensor body 110, or a combination thereof.

When the sensor 102 is deployed within the lumen, either by advancing the sensor 102 out of one or more sheaths, retracting the one or more sheaths, or pulling one or more wires that secure the anchoring elements 500a, 500b and thus the sensor 102 to the tethering delivery system, the anchoring elements 500a, 500b pivot away from the sensor body 110 in substantially opposite directions. For example, the anchoring element 500a can be formed with shape memory material and shaped such that when the anchoring element 500a is released, it pivots to extend in a direction away from the top surface 204, such as to engage the lumen opposite the bottom surface, while the anchoring element 500b is shaped to pivot to extend in an opposite direction than anchoring element 500a. In some embodiments, to achieve the pivot to change the orientation of the anchoring element, the anchoring element may be formed with one or more curve, kink, bend, etc. In some embodiments, when the anchoring elements 500a, 500b pivot away from the sensor body 110 in opposite directions, the sensor body 110 may be held centrally within the lumen. Additionally or alternatively, the anchoring elements 400a, 500b can pivot in the same direction (not shown) to hold the sensor body 110 against a wall of the lumen.

For example, as shown in the top view of FIG. 5B, the anchoring elements 500a, 500b pivot away from the sensor body 110 to engage opposite sides 502a, 502b of the lumen 504. If the anchoring elements 500a, 500b are approximately the same size, the sensor body 110 will be centered within the lumen 504. If the anchoring elements 500a, 500b are different sizes, such that one anchoring element 500a extends a further distance from the sensor body 110 than the anchoring element 500b, the sensor body 110 can be positioned closer to one of the sides 502 of the lumen 504.

In another embodiment, the anchoring elements 500 can be formed to pivot away from the sensor body 110 to engage the same side 502 of the lumen 504. The opposite side of the sensor body 110 would engage the opposite side 502 of the lumen 504 than that engaged by the anchoring elements 500. This configuration can be used to position the sensor body 110 off-center within the lumen 504.

Although the anchoring elements 500 are shown in FIG. 5B as loops, it should be understood that the anchoring elements 500 can be fingers, shaped with protrusions to provide specific points of contact, such as two points of contact, with the lumen 504, etc.

FIG. 5C shows an example wherein anchoring elements 510 are releasably attached to a delivery shaft (not shown) using one or more tether 512 (e.g., release element) in accordance with embodiments herein. For example, the tether 512a can secure the anchoring elements 510a, 510b and the tether 512b can secure the anchoring elements 510c, 510d in a planar configuration along the delivery shaft or system. In other embodiments, a single tether 512 can secure all of the anchoring elements 510a-510d. In still further embodiments, there may only be two anchoring elements 510.

FIGS. 5D and 5E show side and isometric views of the sensor 102 of FIG. 5C deployed within the lumen 504 in accordance with embodiments herein. The anchoring elements 510a, 510b pivot to extend in a first direction, such as in the direction away from the first side surface 208, and the anchoring elements 510c, 510d pivot to extend in an opposite direction, such as in the direction away from the second side surface 210. Although the anchoring elements 510 are shown as loops in FIG. 5E, the anchoring elements 510 can be fingers and/or have one or more protrusion thereon. The anchoring elements 510 engage the opposite sides 502 of the lumen 504 proximate the proximate and distal ends 112, 114 to keep the sensor body 110 centered within the lumen 504.

FIGS. 5F and 5G show side and top views of the sensor 102 having an asymmetric number of anchoring elements 514 coupled to opposite ends of the sensor body 110 in accordance with embodiments herein. The anchoring element 514a is coupled to the distal end 114 of the sensor body 110 and anchoring elements 514b, 514c are coupled to the proximal end 112. It should be understood that the anchoring element 514a can be coupled to the proximal end 112 and the anchoring elements 514b, 514c are coupled to the distal end 114.

When released from the tether or sheath, the anchoring element 514a pivots away from the sensor body 110 to extend in the distal direction 118 and engage the walls of the lumen 504 distal of the sensor body 110. The anchoring elements 514b, 514c pivot away from the sensor body 110 and engage opposite sides of the lumen 504.

Referring to FIG. 5G, the anchoring element 514a having diameter D25 is larger than the anchoring elements 514b, 514c having diameters D26, D27, respectively. In this configuration, the sensor body 110 is held centered within the lumen 504. In other embodiments, the anchoring element 514a can be different sizes than shown to engage a narrowing lumen or a smaller lumen branch, as shown above in FIG. 1E. The anchoring elements 514b, 514c can be the same diameter or different, depending upon whether the sensor body 110 is to be centered within the lumen 504 or positioned off-center.

FIGS. 5H and 51 are top views of the sensor 102 having a portion of the anchoring elements 516 coupled to a central region 520 of the sensor body 110 in accordance with embodiments herein. The central region 520 is located between the proximal and distal ends 112, 114.

Anchoring elements 516a, 516b are coupled to the proximal and distal ends 112, 114, respectively, and when released from the delivery system, the anchoring element 516a pivots away from the sensor body 110 to extend in the proximal direction 116 and the anchoring element 516b pivots away from the sensor body 110 to extend in the distal direction 118. The anchoring elements 516c, 516d are coupled to the sensor body 110 within the central region 520. The anchoring elements 516c, 516d can be coupled to the same side or surface of the sensor body 110, such as the top surface, or to different sides or surfaces. When released from the delivery system, the anchoring elements 516c, 516d pivot away from the sensor body 110 to extend in opposite directions with respect to each other and to engage opposite sides of the lumen.

Referring to FIG. 51, when deployed within the lumen 504, the anchoring elements 516a, 516b have larger diameters D14, D15, respectively, compared to the diameters D16, D17 of the anchoring elements 516a, 516b. The anchoring elements 516 engage the walls of the lumen 504 to keep the sensor body 110 centered within the lumen 504.

FIG. 5J shows a top view of a sensor 102 having anchoring elements 530a, 530b coupled to the sensor body 110 in accordance with embodiments herein. The anchoring elements 530a, 530b can be coupled near the distal and proximal ends 112, 114, or within the central region of the sensor body 110 as discussed in FIG. 5H, to any of the sides of the sensor body 110, and/or any combination thereon.

The anchoring element 530a is substantially triangular, providing one or more rounded points 532a, 532b, each of which will provide a point of contact with the lumen 504. The anchoring element 530b is similarly shaped and has rounded points 532c, 532d that provide points of contact with the lumen 504. In other embodiments, only a portion of the anchoring elements 530 may be shaped with rounded point 532, and in further embodiments, the sensor 102 can include more than two anchoring elements 530 with rounded points 532. In still further embodiments, one or more of the anchoring elements 530 can have more than two rounded points 532, providing additional points of contact with the lumen. In some cases, an advantage can be realized as the triangular shape of the anchoring element 530 can be more difficult to collapse than similarly sized rounded shapes, improving the anchoring within the lumen.

Sensors Having Attachment Feature(s) Integrated with Anchoring Element

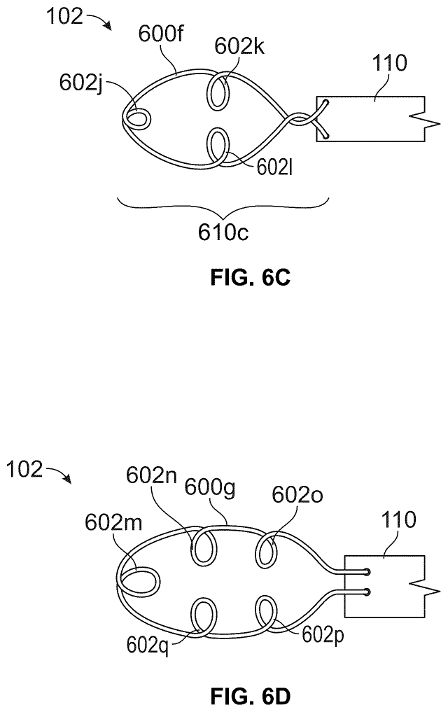

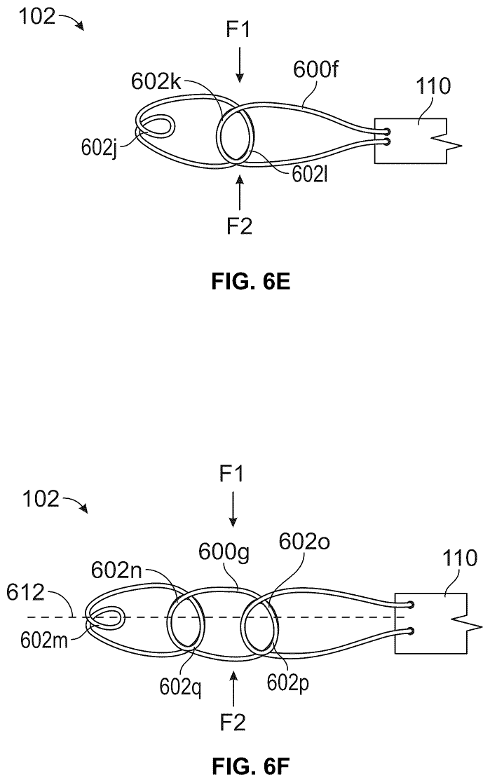

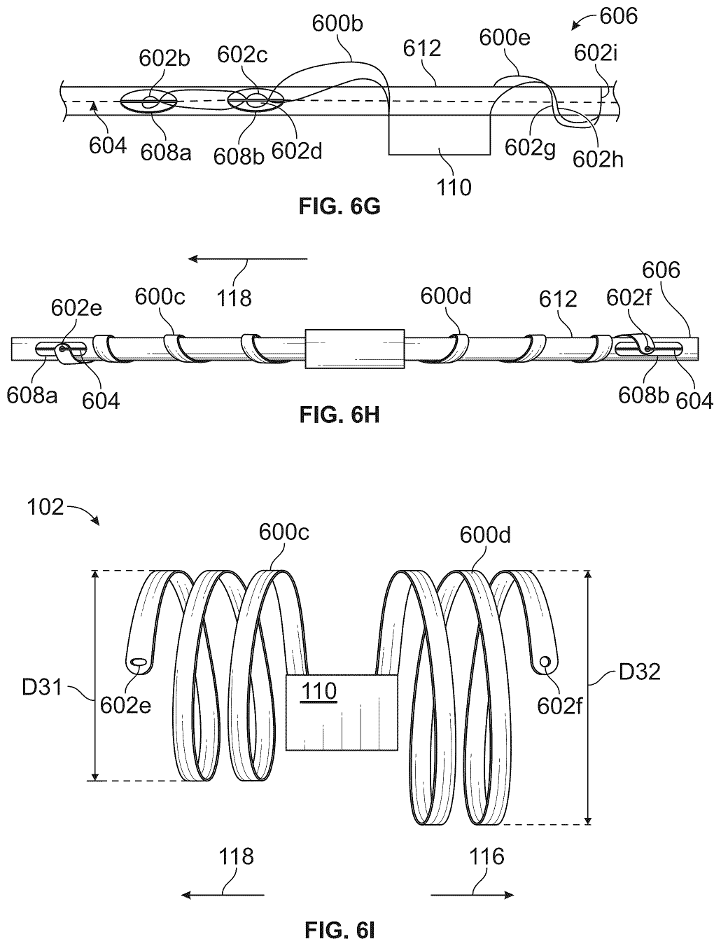

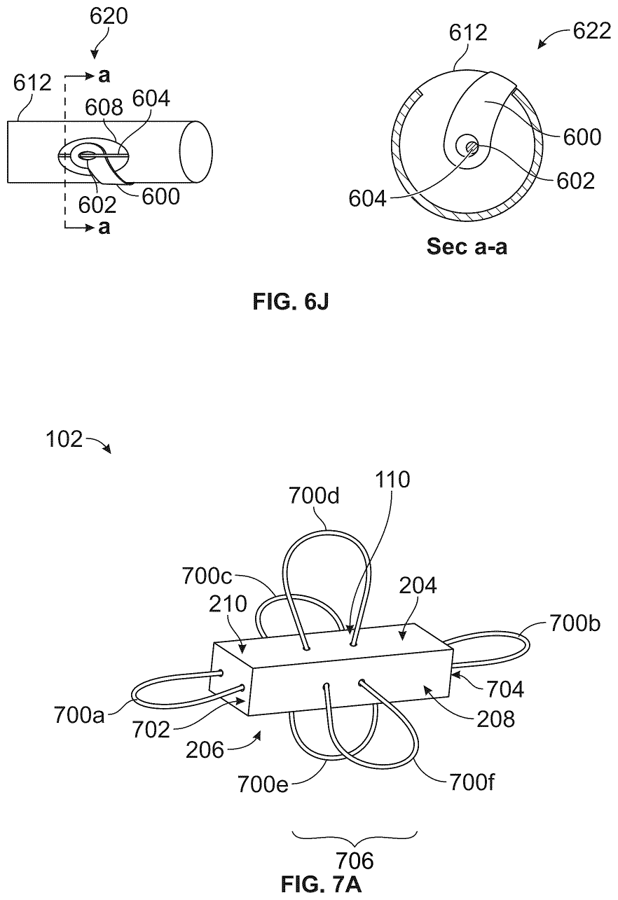

FIGS. 6A and 6B illustrate isometric views of a sensor 102 having an anchoring element that includes at least one integrated attachment feature in accordance with embodiments herein. Although only one anchoring element 600a, 600b is shown coupled to the proximal end 112 of the sensor body 110, another anchoring element can be coupled to the distal end of the sensor body 110, and may or may not have one or more integrated attachment features. Alternatively, the anchoring element 600 can be coupled to the distal end of the sensor body 110.

As shown in FIG. 6A, the anchoring element 600a has an anchoring body 610a that extends in the proximal direction 116 with respect to the proximal end 112 of the sensor body 110. The anchoring body 610a is formed with an integrated attachment feature 602a, which in some embodiments may be an integrated loop made of nitinol or other shape memory material. Referring to FIG. 6B, the attachment features 602b, 602c, 602d are formed integral with the anchoring element 600b within the anchoring body 610b. The attachment features 602c, 602d can be positioned along the anchoring element 600b such that the attachment features 602c, 602d can overlap one another when the different sides of the anchoring element 600b are compressed together. The attachment features 602b, 602c, 602d extend in outward facing loops (e.g., facing toward the wall of the lumen) and can also be anchor points. In some embodiments, the anchoring element 600b can be configured with two attachment features 602 or more than three attachment features 602 that extend outwardly, similar to FIG. 6D below with inward facing loops.

FIG. 6C shows the sensor 102 having the attachment features 602j, 602k, 6021 formed integral with the anchoring element 600f within the anchoring body 610c. The attachment features 602j, 602k, 6021 extend inwardly, resulting in a smooth wire that contacts the lumen wall, which can provide an advantage of minimizing any erosion or damage to the lumen wall.

FIG. 6D also shows attachment features 602m, 602n, 602o, 602p, 602q formed integral with the anchoring element 600g that extend inwardly in accordance with embodiments herein. The attachment features 602m, 602n, 602o, 602p, 602q thus are not configured to directly contact the vessel wall. It should be understood that the number of attachment features 602 can be different than shown (e.g., more than five, an even number of attachment features 602, two, four, or seven attachment features 602). In other embodiments, one or more attachment feature 602 can be formed to face outwardly while one or more attachment feature can be formed to face inwardly with respect to the lumen wall.

FIG. 6E shows the sensor 102 of FIG. 6C wherein the attachment features 602k and 6021 overlap when forces F1 and F2 are applied to compress the anchoring element 600f in accordance with embodiments herein. In some cases, forces F1 and F2 can be in opposite directions with respect to each other. For example, the attachment features 602k, 602l can overlap each other and receive a catheter or sheath, thus encircling an outer surface of the catheter. The attachment features 602 can be sized with respect to a circumference of the catheter, such as to secure the sensor to the catheter, but to also allow the catheter to be removed (e.g., slid through the attachment features 602j, 602k, 602l ) without an undesirable amount of force once the sensor 102 has been positioned within the lumen. It should be understood that the overlap of attachment features 602 can be complete or partial, and that not all attachment features 602 may be the same size or shape.

FIG. 6F shows the sensor 102 of FIG. 6D wherein the attachment features 602n and 602q overlap and attachment features 602o and 602p overlap when forces F1 and F2 are applied to compress the anchoring element 600q in accordance with embodiments herein. Catheter 612 extends through the overlapping holes of the attachment features 602n and 602q and through the overlapping holes of the attachment features 6020 and 602p, as well as through the hole of the attachment feature 602m. In this figure the catheter 612 is represented by a single line for ease of discussion. In other embodiments, a guidewire lumen or a tether can extend through the attachment features 602. In some cases, a tether may extend through less than all of the attachment features 602.

Turning to FIG. 6G, a view of the anchoring element 600b of FIG. 6B is shown retained by a tether 604 to a delivery system 606 in accordance with embodiments herein. The tether 604 extends through a catheter 612 and is retained by the attachment feature 602b at skive 608a. The tether 604 also extends through and is retained by the attachment features 602c, 602d, which have been overlapped at skive 608b. An advantage of including the attachment features 602 on the anchoring element(s) 600 is that the attachments can minimize the number of cut-outs/skives 608 that are needed in the delivery system 606 to retain the sensor 102 with the tether 604. In the example of FIG. 6G, only two skives 608 or openings, located proximate the attachment feature 602b and the attachment features 602c, 602d are needed to releasably retain the anchoring element 600b, and thus the sensor 102, in a fixed relationship with the delivery system 606 in advance of deployment of the sensor 102 in a body. This can simplify the manufacturing process for creating the pieces of the delivery system 606, as well as minimizing the force needed to pull the tether 604 out when releasing the anchoring element(s) 600.

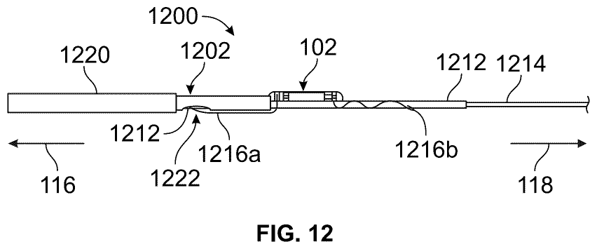

In other embodiments, anchoring element 600e can also include attachment features 602g, 602h, 602i that are similar to the attachment features 602c, 602d, 602b, respectively, of FIG. 6B. The attachment features 602g, 602h, 602i fit around an outer surface of the catheter 612 to secure the sensor body 110 to the catheter 612, and thus can be relatively larger than those that are attached using the tether 604 and skives 608. The use of the attachment features 602g, 602h, 602i prevent the need for twisting the anchoring element 600e around the catheter 612 or guidewire lumen as discussed further below with respect to FIG. 12 that shows the tandem delivery system. In the example of FIG. 6G, the catheter 612 is also a release element. When the catheter 612 is pulled far enough in the proximal direction toward the physician, the anchoring element 600e is free of the catheter 612 and is released. In some cases, this can eliminate the need for skives, creating a smooth release shaft.

FIG. 6H shows coiled ribbon shaped anchoring elements 600c, 600d that can be retained by the tether 604 in accordance with embodiments herein. The anchoring elements 600c, 600d form coiled ribbons having attachment features 602e, 602f, respectively, at either end. The attachment feature 602 can be an eyelet, a hole, a notch, round-shaped, oval-shaped, etc. In the embodiment shown, the anchoring elements 600c, 600d wrap around a catheter 612 of the delivery system 606. The tether 604 can access the attachment feature 602e through skive 608a and extend through the attachment feature 602e, releasably retaining the anchoring element 600c. Similarly, the tether 604 can access the attachment feature 602f through skive 608b and extend through the attachment feature 602f, releasably retaining the anchoring element 600d.

FIG. 61 shows the sensor 102 having the coiled ribbon anchoring element 600c, 600d of FIG. 6H prior to or in advance of being deployed within a lumen in accordance with embodiments herein. The anchoring element 600c, extending in the distal direction 118, has a diameter D31 when released without bias that is smaller than diameter D32 (when released without bias) of the anchoring element 600d that extends in the proximal direction 116. By way of example only, this orientation may be used in pulmonary vessels/lumens that are more narrow in the distal direction 118. The distal and proximal orientations could be reversed such that the anchoring element 600d, having the larger diameter D32, extends in the distal direction 118 to be deployed in peripheral vascular areas where the vessels get larger toward the heart and smaller away from the heart. In some embodiments, the attachment feature 602, such as attachment feature 602e, can be elongated or oval in shape to reduce binding on the release tether. It should be understood that modifying the shape of this feature can be applied to any of the applicable attachment features disclosed herein.

The end of the coiled ribbon anchoring element 600 can be cut or shaped to improve the fit into the skive 608 as shown in FIG. 6J. View 620 is a side view of the catheter 612 that shows the tether 604 extending through the attachment feature 602 of the anchoring element 600 within the skive 608. View 622 is a cross-sectional view of the catheter 612 that shows the anchoring element 600 bent, shaped, cut, etc., such that the anchoring element 600 extends into the skive 608. The anchoring element 600 is positioned such that the tether 604 extends through the attachment feature 602 (e.g., hole) and the catheter 612 with minimal binding, reducing the force needed to release the sensor 102 from the tether 604.

Sensors having Anchoring Elements Attached to Multiple Surfaces

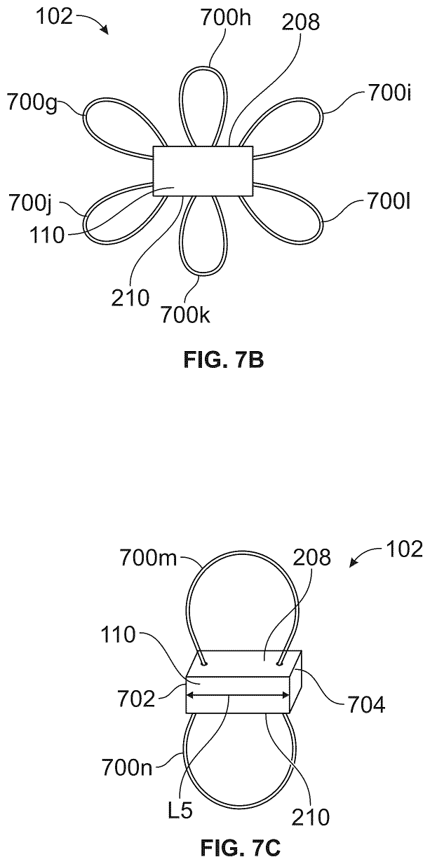

FIG. 7A is an isometric view of the sensor 102 having anchoring elements 700 attached to multiple surfaces in accordance with embodiments herein. The sensor body 110 has top and bottom surfaces 204, 206, first and second side surfaces 208, 210, and first and second end surfaces 702, 704. The anchoring elements 700 are illustrated as loops; however, other shapes can be used. A single anchoring element 700a is coupled to the first end surface 702 and a second anchoring element 700b is coupled to the second end surface 704. The anchoring element 700c is coupled to a central region 706 of the top surface 204, the anchoring element 700d is coupled to the central region 706 of the second side surface 210, the anchoring element 700e is coupled to the central region 706 of the first side surface 208, and the anchoring element 700f is coupled to the central region 706 of the bottom surface 206. By coupling one or more anchoring elements 700 to each of the surfaces, the sensor body 110 is held centered within the lumen. This can be advantageous if the sensor's sensing pad is positioned on the first end surface 702 or the second end surface 704. To position the sensor body 110 off-center within the lumen, anchoring elements 700 can be coupled to conjoining surfaces, such as to the top surface 204 and the first side surface 208. This can be advantageous if the sensor's sensing pad is positioned on one of the top, bottom or side surfaces, such as to position the sensing pad proximate the center of the lumen.

In some embodiments, more than one anchoring element 700 can be coupled to one or more of the outer surfaces of the sensor body 110. In other embodiments, the anchoring elements 700a, 700b can have larger diameters than the anchoring elements coupled to the side surfaces. In still further embodiments, the anchoring elements 700 coupled to the side surfaces can have different diameters to position the sensor body 110 off-center within the lumen.

In some embodiments, the sensor 102 of FIG. 7A can be delivered within the body using a sheathed delivery system, while in other embodiments a tether (not shown) can capture the anchoring elements 700a, 700b to attach the sensor 102 to a tethering delivery system.

FIG. 7B is a top view of a sensor 102 having anchoring elements 700 coupled to opposite side surfaces in accordance with embodiments herein. Anchoring elements 700g, 700h, 700i are coupled to the first side surface 208 and anchoring elements 700j, 700k, 7001 are coupled to the second side surface 210. Coupling anchoring elements 700 to opposite sides of the sensor body 110 retains the sensor body 110 centrally within the lumen, reducing migration or movement of the sensor. If the sensor body 110 is to be centered within the lumen, the size of the anchoring element 700 can be similar or the same with respect to each other. If the sensor body 110 is to be off-center within the lumen, the size of the anchoring elements 700 can be adjusted to achieve the desired amount of off-center placement. For example, the anchoring elements 700g, 700h, 700i can be larger than the anchoring elements 700j, 700k, 7001 if the sensor body 110 is to be closer to the lumen proximate the second side surface 210. In some embodiments, the sensor 102 of FIG. 7B may be used within the vascular system.

FIG. 7C is an isometric view of the sensor 102 having anchoring elements 700 coupled to opposite sides in accordance with embodiments herein. Anchoring element 700m is coupled to the first side surface 208 and anchoring element 700n is coupled to the second side surface 210. The anchoring elements 700m, 700n extend to engage opposite sides of the lumen and may extend beyond a length L5 of the sensor body 110. In other embodiments, the anchoring elements 700m, 700n can be coupled to both of the first and second end surfaces 702, 704 and extend beyond the length L5 of the sensor body 110. The length of the anchoring elements 700m, 700n provides more contact with the lumen compared with smaller loops, thus reducing migration of the sensor 102. The sensor 102 of FIG. 7C can be delivered into the body using the sheathed delivery system, the tethered delivery system, or the tandem delivery system.

Anchoring Element Changes Diameter/Shape with Migration of Sensor

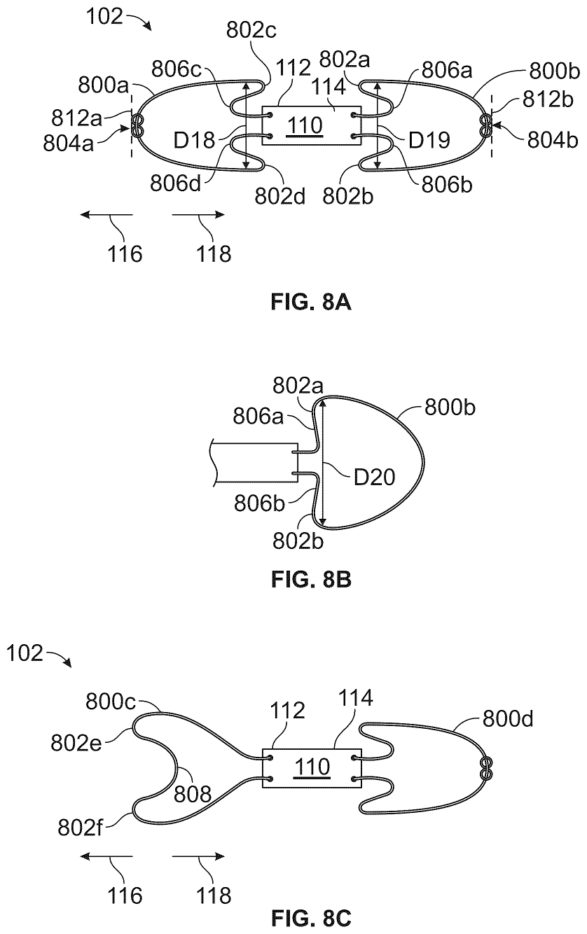

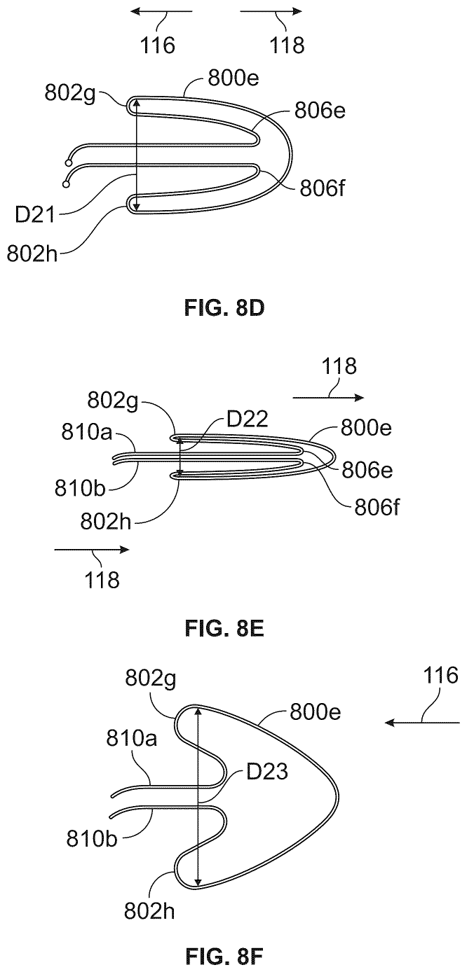

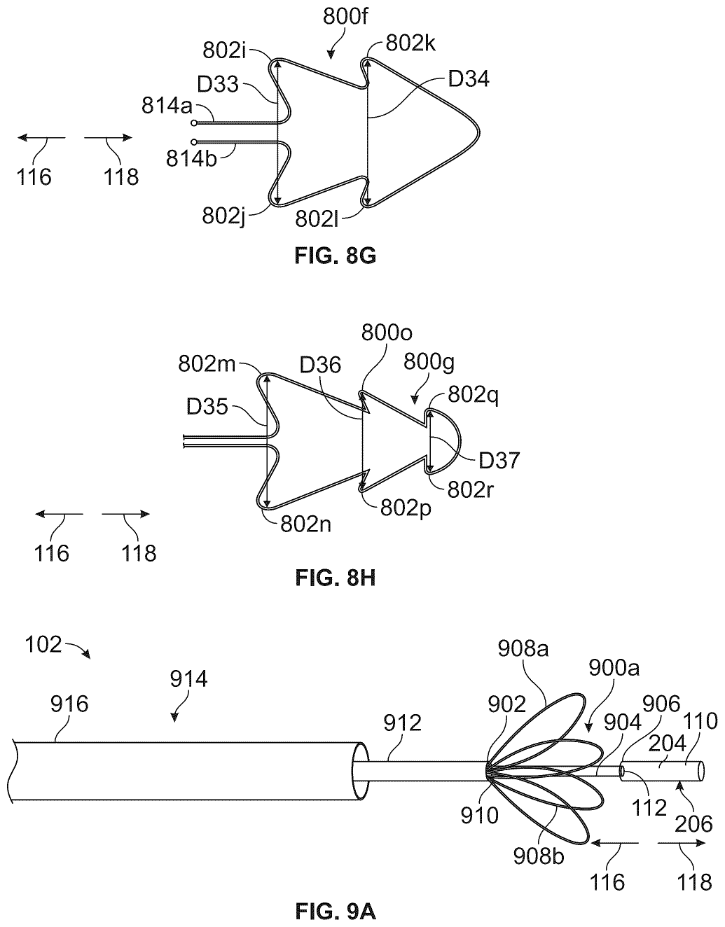

FIGS. 8A-8H include anchoring elements that are configured to alter shape, length, and/or diameter to resist or facilitate movement within the lumen. In some embodiments, the anchoring elements expand as they are allowed by the anatomy, such as to expand to heat set dimensions. In other embodiments, the anchoring elements can expand beyond their heat set dimensions, such as in response to movement of the sensor within the lumen. The expanded dimensions can reduce or prevent the migration of the sensor 102 by remaining engaged with the lumen. FIG. 8A is a top view of a sensor 102 having anchoring elements 800a, 800b coupled to proximal and distal ends 112, 114, respectively, of the sensor body 110 in accordance with embodiments herein. In some embodiments only one of the anchoring elements 800a, 800b may be used.

As shown, the anchoring elements 800a, 800b have an approximate “wing” shape. Anchoring elements 800a, 800b have diameters D18, D19, respectively, indicating the widest point across the anchoring element, and are the same or similar in shape to each other. In some embodiments, the diameter D18, D19 is associated with the diameters of the anchoring elements 800a, 800b when lodged within the lumen. In other embodiments, the diameter D18, D19 is associated with the diameters of the anchoring elements 800a, 800b prior to implant and/or when released without bias. In still further embodiments, the diameters D18 and D19 can be approximately the same with respect to each other, while in other embodiments, the diameters D18 and D19 can be different with respect to each other.

The anchoring element 800b is a loop that includes curved anchor points 802a, 802b that face substantially in the proximal direction 116. Outer portions of the curved anchor points 802a, 802b and side portions of the anchoring element 800b contact opposite sides or walls of the lumen when deployed within the lumen. The anchoring element 800b is bent to form curved portions 806a, 806b that face substantially in the distal direction 118. The loop extends from the curved portions 806a, 806b to couple the anchoring element 800b to the distal end 114 of the sensor body 110.

Due to the shape and orientation of the curved anchor points 802a, 802b, the anchoring element 800b resists movement of the sensor 102 in the proximal direction 116. If the sensor 102 moves in the proximal direction 116, the resistance between the wall of the lumen and the curved anchor points 802a, 802b causes the curved anchor points 802a, 802b and the curved portions 806a, 806b to alter shape, such as to become more flat, resulting in an increased diameter D20 of the anchoring element 800b proximate the curved anchor points 802a, 802b, as shown in FIG. 8B.

The anchoring element 800a is similarly shaped with curved anchor points 802c, 802d that face substantially in the distal direction 118 and curved portions 806c, 806d that face substantially in the proximal direction 116. The anchoring element 800a similarly resists movement in the distal direction 118. The resistance between the wall of the lumen and the curved anchor points 802c, 802d causes the curved anchor points 802c, 802d and the curved portions 806c, 806d to alter shape, such as to flatten, resulting in an increase to the diameter D18 (increase not shown).

As discussed below in FIGS. 8D-8F, migration of the sensor 102 in the proximal direction 116 causes the anchoring element 800a to alter shape to a diameter that is less that the diameter D18, and migration of the sensor 102 in the distal direction 118 causes the anchoring element 800b to alter shape to a diameter that is less than the diameter D19.

In some embodiments, one or both of the anchoring elements 800a, 800b can have an attachment feature 804a, 804b coupled to the loop. The attachment feature 804a, 804b can include one or more loops, holes, notches, etc., and can be attached to the anchoring element 800 through crimping, swaging, etc. As shown, the attachment feature 804a, 804b can be located approximately at a farthest position 812a, 812b proximal and distal, respectively, of the anchoring element 800. A tether (not shown) can extend through the attachment feature(s) 804 to securely couple to sensor 102 to a tethering delivery system. In other embodiments, the sensor 102 can be delivered using a sheathed delivery system.

FIG. 8C is a top view of a sensor 102 having anchoring elements 800c, 800d coupled to proximal and distal ends 112, 114, respectively, of the sensor body 110 in accordance with embodiments herein. The anchoring element 800d can be substantially the same as the anchoring element 800b of FIG. 8A.

The anchoring element 800c is also a “wing” shaped loop having curved anchor points 802e, 802f that substantially point in the proximal direction 116 and a central curved portion 808 that substantially points in the distal direction 118. The anchoring element 800c will alter shape similarly to the anchoring element 800d, altering to have a smaller diameter when the sensor 102 migrates in the distal direction 118, and altering to have a larger diameter when the sensor 102 migrates in the proximal direction 116.

FIGS. 8D, 8E, and 8F are top views of anchoring element 800e in accordance with embodiments herein. The anchoring element 800e has curved anchor points 802g, 802h and curved portions 806e, 806f. Diameter D21, shown in FIG. 8D, is associated with the diameter of the anchoring element 800e when lodged within the lumen, or alternatively, when the anchoring element 800e is released without bias, such as when the anchoring element 800e is formed and prior to being attached to a delivery device.

Turning to FIG. 8E, when implanted, if the anchoring element 800e migrates or is moved in the distal direction 118 in a small lumen, the resistance between the wall of the lumen and the curved anchor points 802g, 802h decreases, causing the curved anchor points 802g, 802h and the curved portions 806e, 806f to alter shape and compress inwardly toward each other and toward legs 810a, 810b that couple the anchoring element 800e to the sensor body 110. In some embodiments, the winged portions of the anchoring element 800e can be compressed to contact one or both of the legs 801a, 801b. The curved portions 806e, 806f are also changed, becoming more narrow. The distal movement results in decreased diameter D22 that is relatively less than diameter D21. The compression allows the anchoring element 800e to be driven into smaller lumens. In other embodiments, the diameter D22 can be a diameter of the anchoring element 800e when collapsed for delivery.

Referring to FIG. 8F, if, when implanted, the anchoring element 800e migrates in the proximal direction 116, the anchoring element 800e alters shape as discussed above with FIG. 8B. The anchor points 802g, 802h are pressed outwardly away from the legs 810a, 810b, resulting in an increased diameter D23, that is relatively greater than the diameter D21.

FIGS. 8G and 8H are top views of anchoring elements 800f, 800g that are configured to alter shape and that have more than two anchor points in accordance with embodiments herein. The anchoring element 800f, 800g are configured to be coupled to a distal end of the sensor body (not shown) with legs 814a, 814b. The anchoring element 800f has curved anchor points 802i, 802k that are positioned to contact a first side of the lumen and curved anchor points 802j, 802l that are positioned to contact a side of the lumen that is opposite the first side. The curved anchor points 802i-802l provide four points of contact with the lumen that provide resistance to movement of the anchoring element 800f in the proximal direction 116, and increase the diameter of the anchoring element 800f. As discussed above, the wings of the anchoring element 800f compress inwardly when the anchoring element 800f moves in the distal direction 118, decreasing the diameter of the anchoring element 800f.

The anchoring element 800g of FIG. 8H has curved anchor points 802m, 8020, 802q that are positioned to contact a first side of the lumen and curved anchor points 802n, 802p, 802r that are positioned to contact a side of the lumen that is opposite the first side. The curved anchor points 802m-802r provide six points of contact with the lumen that provide resistance to movement of the anchoring element 800g in the proximal direction 116 as discussed above. Also, the wings of the anchoring element 800g compress inwardly when the anchoring element 800g moves in the distal direction 118, decreasing the diameter of the anchoring element 800g.

In some embodiments, the diameter when not under bias between the curved anchor points 802 can be approximately the same. For example, in FIG. 8G, diameters D33 and D34 can be approximately the same. In other embodiments, the diameter D33 can be larger than D34, or the diameter D34 can be larger than D33.

In other embodiments, the anchoring element 800 can have a “Christmas tree” shape when not under bias, wherein the diameter at the end further from the sensor body is the narrowest and the diameter at the end closest to the sensor body is the widest. Referring to FIG. 8H, the diameter D35 between the curved anchor points 802m and 802n is greater than the diameter D36 between the curve anchor points 8020 and 802p, and the diameter D36 is greater than the diameter D37 between the curved anchor points 802q and 802r. In still further embodiments, the diameter between the curved anchor points 802 closest to the sensor body can be the narrowest, and the diameters increase further from the sensor body. The tapering of the anchoring element 800 either in the proximal or distal direction, at either end of the sensor body, can be optimized depending upon the anatomy the sensor 102 will be deployed within.

Although a distal anchoring element is shown, it should be understood that similar anchoring elements can be coupled to the proximal end of the sensor body 110 instead of or in addition to the distal anchoring element. Further, embodiments having more than two curved anchor points 802 can be shaped similar to anchoring element 800c of FIG. 8C.

As discussed above in FIGS. 8A-8F, embodiments using the anchoring elements 800f, 800g can be delivered within a body using the tethered delivery system or the sheathed delivery system.

### Anchoring Element Incorporates Rod and is Removably Attachable to Delivery System

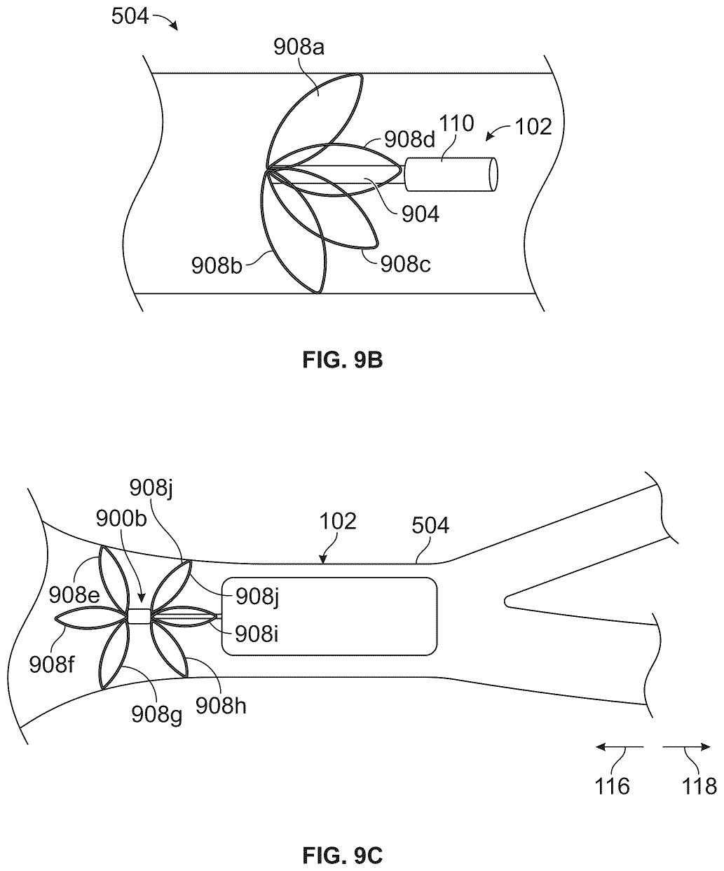

FIGS. 9A-9C show embodiments wherein the anchoring element can also be used as a snare to deliver or retrieve the sensor 102 in accordance with embodiments herein. In FIG. 9A, anchoring element 900a is attached to the proximal end 112 of the sensor body 110, and includes a rod or hollow tube 904 and at least two loops 908a, 908b. The loops 908 are attached to a proximal end 902 of the rod 904. A distal end 906 of the rod 904 is attached to the proximal end 112 of the sensor body 110. Each of the loops 908 are configured to have a different angular orientation such that each of the loops 908 extends at an angle away from the plane that is co-planar between the proximal direction 116 and the distal direction 118 and co-planar with the sensor body 110.

A threaded fastener 910 is included on the proximal end 902 of the rod 904 and is removably attached to a delivery rod 912 of a sheathed delivery system 914. When delivering the assembly into a lumen, an outer sheath 916 contains the sensor 102. The outer sheath 916 holds the loops 908 compressed and tightened around the rod 904, acting as a locking mechanism as the sensor 102 is being delivered. When in the desired location, the sensor 102 is pushed out of the outer sheath 916 in the distal direction 118, or the outer sheath 916 is pulled in the proximal direction 116 to reveal the sensor 102. When released from the outer sheath 916, such as when the outer sheath 616 is retracted in the proximal direction 116 beyond the proximal end 902 of the rod 904, the loops 908 of the anchoring element 900a rotate or pivot outwardly away from the rod 904 to their predetermined shape and orientation to lodge the sensor 102 within the lumen 504. In some embodiments, the loops 908 engage opposite sides of the lumen 504 to hold the sensor body 110 centered within the lumen 504. In other embodiments, the loops 908 engage the lumen 504 to hold the sensor body 110 against the wall of the lumen 504, wherein either the top surface 204 or bottom surface 206 of the sensor body 110 has a more central position within the lumen 504. Once the sensor 102 is positioned within the lumen 504, the delivery rod 912 can be rotated to release the delivery rod 912 from the fastener 910 of the rod 904.

FIG. 9B shows the sensor 102 of FIG. 9A deployed within the lumen 504. The loops 908a, 908b, 908c, 908d are shaped and oriented in a flower or petal configuration, wherein the loops 908a, 908b are shaped and oriented to engage opposite sides of the lumen 504, and the loops 908c, 908d are shaped and oriented to engage two other opposite sides of the lumen 504. Thus, in some embodiments, the loops 908 can engage top, bottom, and both sides of the lumen to hold the sensor body 110 in an approximately central location within the lumen 504.

In some cases, the sensor 102 may need to be removed or moved within the lumen 504 after being deployed. The delivery rod 912 can be reattached to the rod 904, such as by using the threaded fastener 910. The outer sheath 916 can be pushed in the distal direction 118 to cover at least a portion of the loops 908 of the anchoring element 900a. As the outer sheath 916 extends over the loops 908, the loops 908 are compressed to tighten around the rod 904, snaring the sensor 102 as the loops 908 tighten to act as a locking mechanism.

FIG. 9C shows the sensor 102 deployed within the lumen 504 wherein the anchoring element 900b includes a first group of loops 908e, 908f, 908g shaped and oriented to generally extend in the proximal direction 116 and a second group of loops 908h, 908i, 908j shaped and oriented to generally extend in the distal direction 118. The loops 908 are shaped and oriented to contact walls of the lumen 504 and lodge the sensor 102 within the lumen 504. An advantage of having loops 908 that generally extend in the proximal and distal directions 116, 118 is that migration of the sensor 102 in either direction is prevented.

Radiopaque Elements Integrated with Anchoring Body

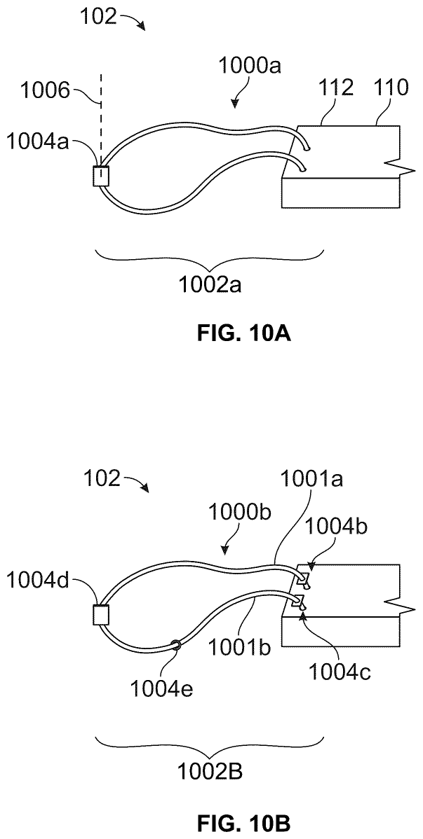

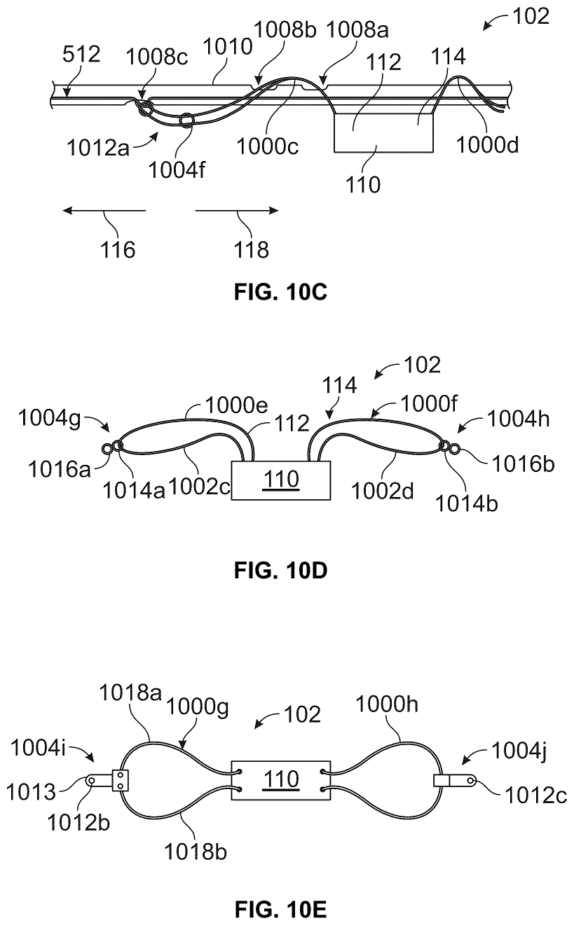

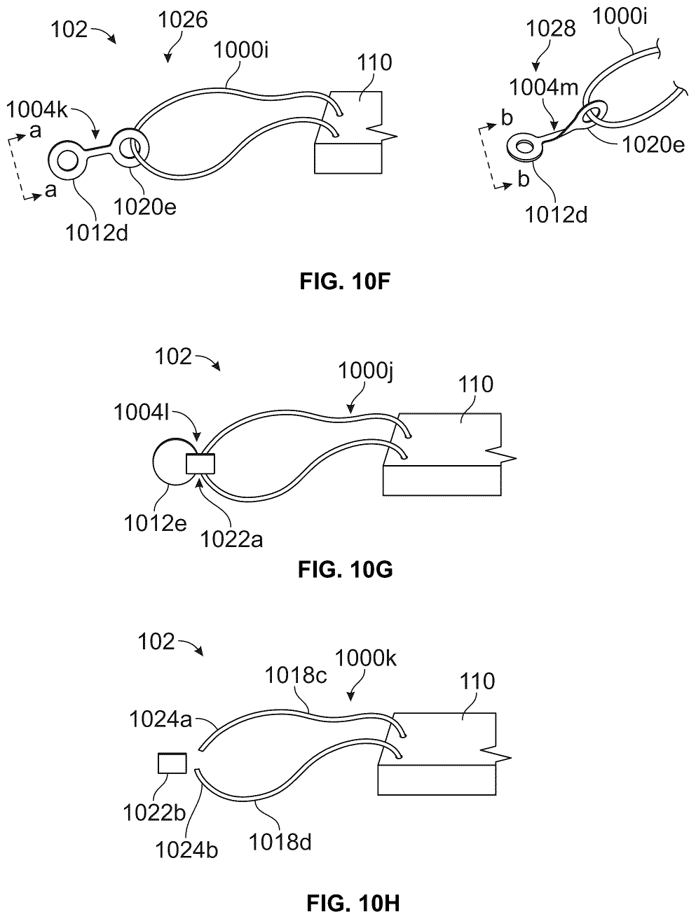

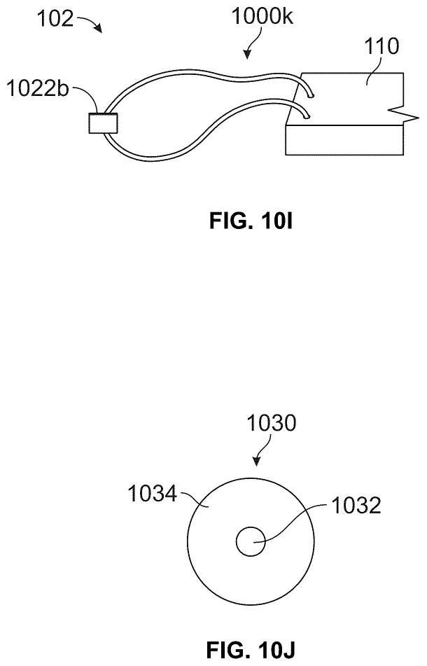

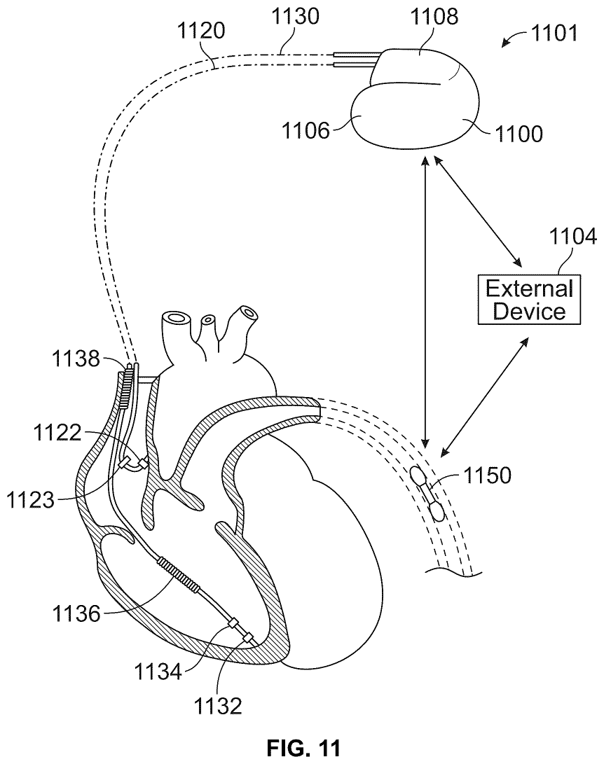

FIGS. 10A-10J show embodiments wherein one or more radiopaque element(s) are integrated with an anchoring body extending away from the sensor body 110 in accordance with embodiments herein. The thin anchor loops are not significantly bright under fluoroscopy or ultrasonic scanning. It can be difficult for a physician or other healthcare worker to see how the anchoring element is engaging the lumen or to see the exact placement of the sensor 102, including the anchoring elements. Although some sensor bodies include a line or race track of radiopaque material that helps the physician see the sensor body 110 during placement, the length the loops or other anchoring shapes extend beyond the sensor body 110 is not known during placement and/or retrieval.

FIG. 10A shows an isometric view of a sensor 102 having an anchoring element 1000a coupled to the proximal end 112 of the sensor body 110.

The anchoring element 1000a has an anchoring body 1002a extending away from the sensor body 110. A radiopaque marker 1004a is integrated with the anchoring body 1002a. The radiopaque marker 1004a can be a swaged markerband and can be made of gold, platinum, iridium, barium filled polymer, tungsten filled polymer or other dense material. In some embodiments, the radiopaque marker 1004a can be attached and/or integrated at a farthest position 1006 of the anchoring element 1000a away from the sensor body 110 to mark a true proximal position or a true distal position (not shown) of the sensor 102. This can beneficially help the physician see the true length of the anchoring element 1000a and thus the true length of the sensor 102.

Although the anchoring element 1000a is shown coupled to the proximal end 112 of the sensor body 110, it should be understood that the anchoring element 1000a or a second anchoring element 1000 can be attached to the distal end 114 of the sensor body 110. An anchoring element 1000 attached to the distal end 114 can have a radiopaque marker 1004 attached to a farthest position of the anchoring element 1000 away from the sensor body 110, marking the true distal position.

FIG. 10B is an isometric view of a sensor 102 having multiple radiopaque markers attached to the anchoring element 1000b. Radiopaque markers 1004b-10004e are shown; however, there may be more or less markers 1004. In some embodiments, the radiopaque markers 1004 can be arranged in a pattern visible under fluoroscopy. The radiopaque markers 1004b, 1004c are attached to the anchoring element 1000b proximate the sensor body 110 where legs 1001a, 1001b of the anchoring element 1000b are retained by the sensor body 110. The radiopaque markers 1004d, 1004e are positioned on the anchoring body 1002b that extends away from the sensor body 110.

FIG. 10C is a side view of a sensor 102 that has attachment features and radiopaque markers in accordance with embodiments herein, and is releasably retained by a tethered delivery system. The sensor body 110 has anchoring element 1000c attached at the proximal end 112 and anchoring element 1000d attached at the distal end 114. The anchoring element 1000c will be discussed, while the anchoring element 1000d can include similar elements and attachment features. The anchoring element 1000c can optionally be fed through openings or skives 1008a, 1008b of a single-lumen or multi-lumen catheter 1010 to enter or exit the catheter 1010. A radiopaque marker 1004f is integrated with the anchoring body of the anchoring element 1000d, such as at a farthest position as discussed above. A distal end of an attachment feature 1012a, such as a polymer tab having a loop, hole, etc., can be integrated with the anchoring body using a secure attachment that can be located at or integral with the radiopaque marker 1004f. The proximal end of the attachment feature 1012a can be pulled to or into skive 1008c and releasably retained by the tether 512 that extends inside the catheter 1010. Although not shown, the radiopaque marker 1004f can be a loop that is also releasably retained by the tether 512 at another skive. The attachment feature 1012a releasably retains the anchoring element 1000c to the delivery system and holds the sensor 102 in a fixed relationship with the tethered delivery system in advance of deployment of the sensor 102 in the lumen. In some cases, the loops and sensor are held, such as by the tether 512, in tight contact with the delivery system. In FIG. 10C and other figures herein, it should be understood that the representation of the anchoring elements and/or associated features (e.g., attachment features, radiopaque markers), that illustrate the elements/features as extending beyond the surface of the catheter and/or guidewire lumen are for illustration and ease of description only. During deployment into a lumen, the sensor 102 and all associated elements/features are releasably attached to the delivery system substantially without slack to hold the components in tight contact with the delivery system.

FIG. 10D is a side view of a sensor 102 having radiopaque markers 1004g, 1004h integrated with the anchoring elements 1000e, 1000f, respectively. Some or all of the material forming the radiopaque markers 1004g, 1004h can be radiopaque. The radiopaque markers 1004g, 1004h include a first loop or hole 1014a, 1014b, respectively, that encircles and retains the anchoring body 1002c, 1002d of the anchoring elements 1000e, 1000f. In some embodiments, the hole 1014a, 1014b can be crimped or otherwise fixed (e.g., mechanical swage, adhesive) to a specific position along the anchoring body 1002c, 1002d or can be free floating. The radiopaque markers 1004g, 1004h include a second integrated attachment feature 1016a, 1016b (e.g., loop or hole) that releasably retains the tether (not shown). In some embodiments, only the portion comprising the first hole 1014a, 1014b or the portion comprising the attachment feature 1016a, 1016b are formed using radiopaque material.

FIG. 10E is a top view of a sensor 102 having different radiopaque markers integrated with anchoring elements 1000g, 1000h. The anchoring element 1000g includes first and second portions 1018a, 1018b. Radiopaque marker 1004i can be a radiopaque coupler that secures the first and second portions 1018a, 1018b together, such as by crimping, swaging, etc. A proximal end 1013 of the radiopaque marker 1004i includes an attachment feature 1012b, such as a loop, hole, etc., that releasably retains the tether (not shown). Radiopaque marker 1004j can be crimped, swaged, or otherwise attached to the anchoring body of the anchoring element 1000h that extends away from the sensor body 110. Integrated attachment feature 1012c, such as a loop, hole, etc., can releasably retain the tether. When the tether is extended through the integrated attachment features 1012b, 1012c, the sensor 102 is held in a fixed relationship with the tethered delivery system (not shown) in advance of deployment of the sensor 102 in the lumen.

FIGS. 10D and 10E show embodiments wherein the hole orientation of the integrated attachment feature 1012, 1016 that releasably retains the tether is in approximately the same plane as the loop 1014 or other coupler that interfaces with/attaches to the anchoring element. FIG. 10F shows a view wherein the hole orientation of the integrated attachment feature that releasably retains the tether is approximately in the same plane as the loop that interfaces with the anchoring element and another view wherein the hole orientation is twisted, such as to optimize the profile to the delivery system, in accordance with embodiments herein. View a-a 1026 shows a view of a sensor 102 with a single piece of radiopaque material that forms another radiopaque marker 1004k. Radiopaque marker 1004k includes an attachment feature 1020e (e.g., hole) that retains the anchoring element 1000i and an attachment feature 1012d that releasably retains the tether (not shown) The attachment feature 1020e and the attachment feature 1012d are approximately in the same plane. View b-b 1028 shows a view of the anchoring element 1000i and radiopaque marker 1004m without the sensor body 110. The attachment feature 1020e and the attachment feature 1012d are “twisted” or oriented with respect to each other such that the features 1020e and 1012d are not in the same plane. The attachment feature 1012d can be twisted, rotated, oriented, etc., in either clock-wise or counter clock-wise directions with respect to the attachment feature 1020e to achieve a desired profile to the delivery system, such as to align with a skive, extend into a skive, facilitate smoother release of the tether with less resistance, etc. It should be understood that features of other embodiments herein may similarly be oriented in different planes to optimize the profile to the delivery system.

FIG. 10G shows a view of a sensor 102 with a radiopaque coupler and an attachment feature integrated with the anchoring element 1000j. Radiopaque marker 10041 includes an attachment feature 1012e that can be a loop that releasably retains the tether (not shown). In some embodiments, the attachment feature 1012e can be a shape memory material such as nitinol, a polymer or any other shape memory material known in the art, or can be stainless steel, cobalt chromium, barium or tungsten filled polymer, etc. A radiopaque coupler 1022a joins attachment feature 1012e and the anchoring element 1000j, which can also, in some embodiments, be formed of nitinol.

FIGS. 10H and 10I show coupled and uncoupled views, respectively, of a sensor 102 that has a two-part anchoring element joined with a radiopaque coupler. Anchoring element 1000k has two portions 1018c, 1018d. A radiopaque coupler 1022b can be crimped onto outer ends 1024a, 1024b of each of the portions 1018c, 1018d, to join them together. In some embodiments, the radiopaque coupler 1022b is positioned at or approximate the farthest position away from the sensor body 110.

FIG. 10J is a cross-sectional view of a wire 1030 used to form an anchoring element in accordance with embodiments herein. The wire 1030 has a radiopaque core 1032 at least partially surrounded by another layer of material 1034, such as any appropriate shape memory material. In some embodiments, the radiopaque core 1032 may extend through all or portions of the anchoring body of the anchoring element that extends away from the sensor body 110.

In other embodiments, radiopaque material can extend over all or portions of an outer surface of the anchoring element 1000. For example, all of portions of the outer surface of the anchoring element 1000 can be coated, dipped, plated, sputter coated, gilded, plated, etc., with the radiopaque material.

### System Utilizing Implantable Sensor

FIG. 11 illustrates a system 1101 that includes an implantable medical device (IMD) 1100, an implantable sensor 1150, and an external device 1104 implemented in accordance with embodiments herein. The IMD 1100 and the implantable sensor 1150 (e.g., the sensor 102 described above) are implanted within the body of a patient. The external device 1104 is outside of the patient body. The external device 1104 may be a programmer, an external defibrillator, a workstation, a portable computer (e.g., laptop or tablet computer), a personal digital assistant, a cell phone (e.g., smartphone), a bedside monitor, a wand, a pillow, or other product that the patient can lay against and/or rest against their body, and the like. The IMD 1100 may represent a cardiac monitoring device, a pacemaker, a cardioverter, a cardiac rhythm management device, a defibrillator, a neurostimulator, a leadless monitoring device, a leadless pacemaker, and the like, implemented in accordance with one embodiment of the present invention. The IMD 1100 may be a dual-chamber stimulation device capable of treating both fast and slow arrhythmias with stimulation therapy, including cardioversion, defibrillation, anti-tachycardia pacing and pacing stimulation, as well as capable of detecting heart failure, evaluating its severity, tracking the progression thereof, and controlling the delivery of therapy and warnings in response thereto.

The IMD 1100 includes a housing 1106 that is joined to a header assembly 1108 that holds receptacle connectors connected to a right ventricular lead 1130 and an atrial lead 1120, respectively. The atrial lead 1120 includes a tip electrode 1122 and a ring electrode 1123. The right ventricular lead 1130 includes an RV tip electrode 1132, an RV ring electrode 1134, an RV coil electrode 1136, and an SVC coil electrode 1138. The leads 1120 and 1130 detect intracardiac electrogram (IEGM) signals that are processed and analyzed, and also deliver therapies.

The IMD 1100 may be implemented as a full-function biventricular pacemaker, equipped with both atrial and ventricular sensing and pacing circuitry for four chamber sensing and stimulation therapy (including both pacing and shock treatment). Optionally, the IMD 1100 may further include a coronary sinus lead with left ventricular electrodes. The IMD 1100 may provide full-function cardiac resynchronization therapy. Alternatively, the IMD 1100 may be implemented with a reduced set of functions and components. For instance, the IMD may be implemented without ventricular sensing and pacing.

The implantable sensor 1150 is configured to be implanted at a location remote from the electrodes of the leads 1120 and 1130. The implantable sensor 150 may be implanted in a blood vessel (e.g., lumen), such as an artery or vein. In an embodiment, the sensor 1150 is implanted within the pulmonary artery (PA). The sensor 1150 may be anchored to the vessel wall of a blood vessel or lumen using one or more expandable anchoring elements. The diameter of each anchoring element should be larger than the diameter of target blood vessel/lumen in order to provide adequate anchoring force.

The sensor 1150 is configured to sense a physiologic parameter of interest (PPOI) and to generate signals indicative of the PPOI. In a non-limiting example, when the sensor 1150 is disposed within the PA, the sensor 1150 may sense, as the PPOI, blood pressure.

In some embodiments, the sensor 1150 can be powered by, and communicate with, the external device 1104. In other embodiments, the sensor 1150 can communicate with the IMD 1100. The sensor 1150 can communicate information about the physiologic parameter, for example.

### Delivery Systems

By way of example, FIGS. 4A-4C and 9A show exemplary sheathed delivery system(s), and portions of FIGS. 6G, 6H and 10C show exemplary tethering delivery systems. FIG. 12 is an exemplary tandem delivery system 1200. A delivery catheter 1202 is positioned in tandem to the sensor 102, and may include an optional pressure sheath 1220. A guidewire lumen (GWL) 1212 extends within the catheter 1202, such as within one lumen of the multi-lumen catheter or within the single lumen catheter. A guidewire 1214 can extend within the GWL 1212. One or both of the guidewire 1214 and the GWL 1212 can be configured as a release element.

The sensor 102 can utilize one or more anchoring elements 1216a, 1216b, such as loops, to removably couple the sensor 102 to the sensor delivery system 1200. The anchoring element 1216b interfaces with an outer surface of the GWL 1212, such as with an interlacing weave pattern, twisting, wrapping, etc. A portion of the anchoring element 1216a can be retained between the GWL 1212 and the catheter 1202. The anchoring element 1216a can extend into and/or through an opening or skive 1222 in the catheter 1202, be positioned between the GWL 1212 and an inner portion of the catheter 1202 and/or the skive 1222, and exit through the skive 1222 on an opposite side of the GWL 1212. Therefore, the sensor 102 is secured and/or interlocked to the catheter 1202 while the GWL 1212 extends through the portion of the catheter 1202 coinciding with the skive 1222 and the proximal loop 1218.

All references, including publications, patent applications and patents, cited herein are hereby incorporated by reference in their entireties to the same extent as if each reference were individually and specifically indicated to be incorporated by reference and were set forth in its entirety herein.

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An implantable wireless sensor for deploying within a lumen in a body, comprising:
a sensor body comprising a proximal end and a distal end; and
a first anchoring element coupled to the proximal end of the sensor body and a second anchoring element coupled to the distal end of the sensor body, at least one of the first and second anchoring elements configured to lodge the sensor body within the lumen, wherein the first and second anchoring elements are asymmetric by i) size with respect to each other prior to releasably retaining the sensor in a fixed relationship with a delivery system or ii) number of elements.

2. The sensor of claim 1, wherein the first anchoring element coupled to the proximal end of the sensor body is relatively larger than the second anchoring element coupled to the distal end of the sensor body.

3. The sensor of claim 1, wherein the first anchoring element coupled to the proximal end of the sensor body spans a larger diameter than the second anchoring element, wherein the first anchoring element is configured to lodge in a relatively larger lumen and the second anchoring element is configured to lodge in a relatively smaller lumen.

4. The sensor of claim 1, wherein the second anchoring element comprises two elements, wherein the two elements comprise first and second loops, wherein the first loop is oriented to extend at a non-zero angle with respect to at least one of horizontal and vertical planes associated with the sensor body, wherein each of the first and second loops span smaller diameters than the first anchoring element to lodge in second and third lumens extending from the first lumen that are relatively smaller than the first lumen.

5. The sensor of claim 1, wherein the first anchoring element is a body anchoring loop and the second anchoring element is a distal anchoring loop, wherein the body anchoring loop is relatively longer than the distal anchoring loop, the body anchoring loop and distal anchoring loop extending distally with respect to the sensor body, a distal end of the body anchoring loop extending under the distal anchoring loop, wherein the body anchoring loop is configured to engage the lumen proximate opposite first and second side surfaces of the sensor.

6. The sensor of claim 1, wherein the first anchoring element is a body anchoring loop and the second anchoring element is a proximal anchoring loop, wherein the body anchoring loop is relatively longer than the proximal anchoring loop, the body anchoring loop and proximal anchoring loop extending proximally with respect to the sensor body, a proximal end of the body anchoring loop extending under the proximal anchoring loop, wherein the body anchoring loop is configured to engage the lumen proximate opposite first and second side surfaces of the sensor.

7. The sensor of claim 6, wherein the first and second anchoring elements are configured to extend coplanar with proximal and distal directions.

8. The sensor of claim 6, wherein the first and second anchoring elements include distal portions proximate the distal end of the sensor body, the sensor further comprising a restraining feature coupled to the distal end of the sensor body, the restraining feature configured to restrain the distal portions of the first and second anchoring elements.

9. An implantable wireless sensor for deploying within a lumen in a body, comprising:
a sensor body comprising a proximal end and a distal end; and
a first anchoring element coupled to the proximal end of the sensor body and a second anchoring element coupled to the distal end of the sensor body, at least one of the first and second anchoring elements configured to lodge the sensor body within the lumen,
wherein the first anchoring element comprises an anchoring body extending in a proximal direction with respect to the proximal end of the sensor body, wherein the anchoring body includes at least one integrated attachment feature configured to retain a release element associated with a delivery system, wherein the release element is configured to releasably retain the sensor in a fixed relationship with a delivery system in advance of deployment of the sensor in a body.

10. The sensor of claim 9, wherein the at least one integrated attachment feature is a loop.

11. The sensor of claim 9, wherein the at least one integrated attachment feature comprises first, second, and third integrated attachment features configured to retain the release element, the first and second integrated attachment features positioned on opposite sides of the anchoring body, the first and second integrated attachment features positioned to overlap each other when retained by the release element.

12. An implantable wireless sensor for deploying within a lumen in a body, comprising:
a sensor body comprising a proximal end and a distal end; and
a first anchoring element coupled to the distal end of the sensor body, the first anchoring element configured to lodge the sensor body within the lumen, the first anchoring element comprising a loop having a first diameter when the first anchoring element is freely released and not subject to any bias,
responsive to a force moving the sensor body in a distal direction within the lumen after the sensor is deployed within the lumen, the first anchoring element is configured to alter shape to a second diameter that is less than the first diameter,
responsive to the sensor body moving in a proximal direction within the lumen after the sensor is deployed within the lumen, the first anchoring element is configured to alter shape to a third diameter that is greater than the first diameter.

13. The sensor of claim 12, wherein the first anchoring element is shaped to form two curved anchor points facing in the proximal direction when having the first diameter.

14. The sensor of claim 13, wherein the two curved anchor points are positioned to contact opposite sides of the lumen.

15. The sensor of claim 12, wherein the first anchoring element is shaped to form at least four curved anchor points facing in the proximal direction when having the first diameter, wherein a first portion of the at least four curved anchor points are positioned to contact a first side of the lumen and a second portion of the at least four curved anchor points are positioned to contact a second side of the lumen that is opposite the first side of the lumen.

16. The sensor of claim 12, further comprising a second anchoring element coupled to the proximal end of the sensor body, the second anchoring element configured to lodge the sensor body within the lumen, the second anchoring element comprising a loop having a fourth diameter when the second anchoring element is lodged within the lumen, wherein the second anchoring element is shaped to form at least two curved anchor points facing in the distal direction.

17. The sensor of claim 16, wherein responsive to the sensor body moving in the proximal direction within the lumen after the sensor is deployed within the lumen, the second anchoring element is configured to alter shape to a fifth diameter that is less than the fourth diameter, responsive to the sensor body moving in the distal direction within the lumen after the sensor is deployed within the lumen, the second anchoring element is configured to alter shape to a sixth diameter that is greater than the fourth diameter.
